(19) United States
(12) Patent Application Publication
(10) Pub. No.: US 2022/0256821 A1
(43) Pub. Date: Aug. 18, 2022

(54) ZEBRAFISH DELETION AND COMPOUND MUTANTS AND USES THEREOF

(71) Applicant: The General Hospital Corporation, Boston, MA (US)

(72) Inventors: David M. Langenau, Stoneham, MA (US); Yan Chuan, Boston, MA (US)

(21) Appl. No.: 17/626,728

(22) PCT Filed: Jul. 14, 2020
(86) PCT No.: PCT/US2020/041971

§ 371 Date: Jan. 12, 2022

Related U.S. Application Data
(60) Provisional application No. 62/874,075, filed on Jul. 15, 2019.

(51) Int. Cl. A01K 67/027; G01N 33/50; A61K 49/00

(52) U.S. Cl. CPC A01K 67/0276; G01N 33/5088; A61K 49/006; A01K 2267/0393; A01K 2217/075; A01K 2227/40; A01K 2267/0331; A01K 2207/12

(57) ABSTRACT

Genetically-modified zebrafish lacking one or more immune-related genes, and the use thereof, e.g., in cell or tissue transplantation methods or in stem cell biology. Tumors, tissues, and cells originating from zebrafish, other fish species, frogs, mouse, human, or other mammals can be readily engrafted into zebrafish that lack specific immune system regulatory genes. Here, described are zebrafish in which the entire genomic regions comprising the coding sequences of genes required for the development of T, B, and NK cells (including NK-lysin expressing cells) are deleted.

### CLAIM OF PRIORITY

This application claims the benefit of U.S. Provisional Patent Application Ser. No. 62/874,075, filed on Jul. 15, 2019. The entire contents of the foregoing are hereby incorporated by reference.

### FEDERAL FUNDING

This invention was made with government support under Grant. No. OD016761 awarded by the National Institutes of Health. The government has certain rights in the invention.

### TECHNICAL FIELD

This invention relates to genetically-modified zebrafish lacking one or more immune-related genes, and the use thereof, e.g., in cell or tissue transplantation methods, in stem cell biology, cancer modeling, and drug screening.

### BACKGROUND

Cell transplantation is an important experimental tool to define mechanisms that drive continued tumor growth, metastasis, and therapy responses. Using these approaches, it is now possible to xenograft human cancers into immune deficient mice, including the current gold standard—NOD.Cg-PrkdC^{scid} Il2rg^{tm1Wjl/SzJ} (NSG) mice. Despite the ever-increasing diversity of mouse xenoengraftment models and more sophisticated approaches to humanize mice, there are inherent limitations to the mouse model. For example, mice are expensive and require large vivarium space—limiting the scale of experimentation. Mice are also furred and imaging through the skin is difficult. In fact, single cell imaging modalities largely focus on creating surgical windows and utilizing complex multi-photon imaging modalities to see deeply into organs and tissues. Because these windows must be created prior to implantation of tumor, it is not possible to image disseminated cancer cells throughout the whole animal.

Deletion mutants for genes described herein have not been reported in the literature. The indel mutation in il2rgb has not been reported in any manuscript to date. Generation and use of il2rga (a different gene) has been previously published in Tang et al, J Exp Med. 2017 Oct. 2; 214(10):2875-2887 (PMID: 28878000) and Yan et al., Cell. 2019 Jun. 13; 177(7):1903-1914 (PMID: 31031007). The il2rga allele has not been reported to be complexed with the newly generated deletion alleles or the il2rgb^{D33fs} mutant. The rag2 and NK-lysin deletion mutations have not been reported in the literature nor been reported to be complexed with frameshift mutations including il2rga, il2rgb^{D33fs}, or prkdc^{D3612fs}.

New models for cell transplantation are needed.

### SUMMARY

Capitalizing on the transparent nature of zebrafish, described herein are mutant zebrafish lines that have impaired immune cell function resulting from genetic mutations in one or more immune-related genes.

Tumors, tissues, and cells originating from zebrafish, other fish species, frogs, mouse, human, or other mammals can be readily engrafted into zebrafish that lack specific immune system regulatory genes. Here, described are zebrafish in which the entire genomic regions comprising the coding sequences of genes required for the development of T, B, and NK cells (including NK-lysin expressing cells) are deleted. Specially, described herein are fish generated via genome engineering methods targeting the 5′ and 3′ ends of genes, with intervening DNA sequences being excised following non-homologous end joining. These methods create zebrafish with deletion/null mutations in key regulatory regions in genes required for immune system function. These animals are bred to generate viable, compound mutant fish.

The optically-clear, homozygous compound mutant fish described herein (e.g., the rag2^{null/null}, il2rga^{Y91fs} casper-strain zebrafish) can be engrafted with human cancers, APECs, and T cells to dynamically visualize T cell killing of cancer cells within the fish.

This application also describes a new allele for the il2rgb locus.

This application also provides a list of additional genes that are targeted for deletion in zebrafish.

The techniques described herein cut out part or all of the genomic DNA sequence coding for the functional protein (rather than using a single genome engineering event and indel mutation that results in truncated proteins due to DNA frame shift/stop mutations). The mutant zebrafish described herein have deleted regulatory and/or whole coding sequences of important immune cell genes; these fish may be complexed with additional deletion, point mutant, and indel mutant animals to create compound mutant fish with mutations in multiple genes.

The fish described herein are useful for engrafting tissues originating from zebrafish, other fish species, frogs, mouse, human, or other mammals. Tissues include cancers, regenerative tissues, ES, IPSCs, and blood. Such models are useful for preclinical modeling of therapy responses and also for single cell imaging of responses to genetic and chemical modification of either engrafted cells or the stroma of the immune deficient zebrafish.

In some aspects, provided herein is a genetically-modified fish whose genome is homozygous for a first engineered or induced genetic alteration in recombination-activating gene 2 (rag2) and for a second engineered or induced genetic alteration in interleukin 2-receptor gamma a (il2rga); wherein the first genetic alteration results in an inactivation of both alleles of rag2; wherein the second genetic alteration results in an inactivation of both alleles of il2rga; and wherein the genetic alteration in rag2 is amorphic. In some aspects, the genetically-modified fish has a genotype rag2^{null/null}; il2rga^{Y91fs}−/−.

In some aspects, provided herein is a genetically-modified fish whose genome is homozygous for an engineered or induced genetic alteration in interleukin 2-receptor gamma b (il2rgb); wherein the genetic alteration results in an inactivation of both alleles of il2rgb. In some aspects, the genetically-modified fish has a genotype il2rgb^{D33fs}−/−.

In some aspects, provided herein is a genetically-modified fish whose genome is homozygous for an engineered or induced genetic alteration in NK-lysin; wherein the genetic alteration results in an inactivation of both alleles of NK-lysin. In some aspects, the genetically-modified fish has a genotype NK-lysin^{null/null}.

In some aspects, provided herein is a genetically-modified fish whose genome is homozygous for a first engineered or induced genetic alteration in protein kinase, catalytic subunit-deficiency (prkdc), for a second engineered or induced genetic alteration in Nk-lysin genes nkla, nklb, nklc, and nkld, for a third engineered or induced genetic alteration in interleukin 2-receptor gamma a (il2rga), and for a fourth engineered or induced genetic alteration in interleukin 2-receptor gamma b (il2rgb); wherein the first genetic alteration results in an inactivation of both alleles of prkdc; wherein the second genetic alteration results in an inactivation of both alleles of the Nk-lysin genes nkla, nklb, nklc, and nkld; wherein the third genetic alteration results in an inactivation of both alleles of il2rga; and wherein the fourth genetic alteration results in an inactivation of both alleles of il2rgb. In some aspects, the genetically-modified fish has a genotype of prkdc^{D3612fs}−/−; NK-lysin^{null/null}; il2rga^{Y91fs}−/−; il2rgb^{D33fs}−/−.

In some aspects, the genetically-modified fish described herein are tolerant to irradiation.

In some aspects, provided herein is a method of growing a mammalian cell, the method comprising transplanting the mammalian cell into a genetically-modified fish described herein. In some aspects, the mammalian cell is a tumor cell. In some aspects, the cell is a stem cell or progeny of differentiated stem cell. In some aspects, the cell is a T cell. In some aspects, the cell is a blood cell.

In some aspects, provided herein is a method of identifying a candidate therapeutic compound for the treatment of a mammalian tumor, the method comprising: transplanting cells from a mammalian tumor into a genetically-modified fish described herein; contacting the fish with a test compound; evaluating the growth of a tumor comprising the mammalian tumor cells in the presence of the test compound; comparing the level of growth of the tumor in the presence of the test compound to a level of growth of a tumor in the presence of control substance; and identifying a compound that decreases the level growth of the tumor as a candidate therapeutic compound. In some aspects, the mammalian tumor cells are from a subject with cancer, and the method further comprises administering the identified candidate therapeutic compound to the subject. In some aspects, the method further comprises transplanting mammalian T cells into the genetically-modified fish. In some aspects, the therapeutic compound is a chimeric antigen receptor (CAR), a bispecific T cell engager (BiTE), or an antibody peptide epitope circuit therapy (APEC). In some aspects, the method further comprises transplanting mammalian chimeric antigen receptor T cells into the genetically-modified fish.

Also provided herein is a method of detecting an effect of a test compound on development of a cell or tissue, the method comprising: transplanting a stem or progenitor cell into a genetically-modified fish described herein; contacting the fish with a test compound for a time sufficient for the stem or progenitor cell to develop; evaluating the development of the stem or progenitor cell or its progeny in the presence of the test compound; comparing the development of the stem or progenitor cell or its progeny in the presence of the test compound to development of the stem or progenitor cell or its progeny in the absence of the test compound; and identifying an effect of the test compound on development of the stem or progenitor cell or its progeny. In some aspects, the stem or progenitor cell is labeled. In some aspects, identifying an effect of the test compound on development of the stem or progenitor cell or its progeny comprises visualization of the cells in vivo or sectioning and staining the cells. In some aspects, the test compound is a drug or genetic modification. In some aspects, the stem or progenitor cell is a hematopoietic stem cell.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A-FIG. 1E: rag2^{null/null}; il2rga−/− immunocompromised zebrafish robustly engraft human cancers and normal, non-transformed T cells. FIG. 1A: Schematic of 3.3 kb genome deletion in the rag2 gene locus, with starred boxes indicating genomic sequences targeted by the CRISPR/Cas9 guide RNAs (top). Target sequences of guide RNA targeting the rag2 gene (bottom). SEQ ID NOs: 1 (top left), 2 (top right), 3 (bottom left), 4 (bottom right). FIG. 1B: Indrop single cell RNA sequencing analysis of wild type (WT) (top), rag2^{null/null} (middle) and rag2^{null/null}; Il2rga−/− (bottom), with compounded mutant animals having vastly reduced mature T, B, and NK cell populations. FIG. 1C: Representative images of rag2^{null/null}; Il2rga−/− adult immunocompromised zebrafish engrafted with GFP-expressing rhabdomyosarcoma (RD; top row), cholangiocarcinoma (SNU11196; middle row) and small cell lung cancer (NCI-H86; bottom row) human cancer cell lines, imaged on day 0 and 28 of engraftment, as well as hematoxylin and eosin staining of engrafted tumor sections at the end of the experiment (right panels). FIG. 1D: Summary of engraftment rates comparing the previously reported prkdc−/−, il2rga−/− model and the rag2^{null/null}; Il2rga−/− model. The rag2^{null/null}; Il2rga−/− model engrafts MCAS, OVCAR-5, NCI-H83, 1518-B3 P6 M58, 1526-2A5 P7 M10, SNU1196, and AZD1175 human cancer cells significantly better than the previously reported prkdc−/−, il2rga−/− zebrafish (p<0.05 by Fisher Exact Test, number of engrafted fish/total fish denoted). FIG. 1E: Flow cytometry analysis of human CD8+ T cells engraftment. Human CD8+ T cells were stained with CSFE, intra-peritoneally transplanted, and recipient zebrafish peripheral blood extracted at day 1, 7 and 14 for analysis.

FIG. 1F: Homozygous rag2^{null/null}, il2rga−/− casper-strain zebrafish survive to adulthood and engraft a wide array of human cancers. GFP-labeled human rhabdomyosarcoma (RD), breast cancer (MDA-MB-231), and ovarian cancer (MCAS) efficiently engraft into 2-4 month-old rag2^{null/null}, 2rga−/− fish for up to 21 days post transplantation. Number of successfully engrafted fish shown in upper left and quantitation of growth in successfully engrafted animals (right graphs). Irradiation was not used in these engraftment procedures.

FIG. 1G: Homozygous rag2^{null/null}il2rga−/− capser-strain zebrafish can efficiently engraft human peripheral mononuclear blood cells (PMBCs) in vivo. rag2^{null/null}, il2rga−/− were engrafted with human CSFE-labeled PBMCs and then injected into rag2^{null/null}, il2rga−/− fish that had received 20 gy irradiation 2 days prior to implantation. FACs was used to assess fluorescent-labeled blood cells harvested after 14 days of engraftment. Representative FACs plots from three separate engrafted fish.

FIG. 1H: Homozygous rag2^{null/null}il2rga−/− casper strain zebrafish survive to adulthood and can be irradiated. rag2^{null/null}, il2rga−/− were created by incrossing rag2^{null/+}, il2rga−/+ animals and assessed for ability to survive to 3 months of age. These fish are generated at the expected Mendelian ratios when scale genotyped at 3 months of age and withstand 20 gy gamma-irradiation, which would likely be required to clear the marrow niche for blood cell transplants. In comparison, prkdc−/− or prkdc−/−, il2rga−/− zebrafish do not survive the irradiation procedure. N=10 animals/group.

FIG. 2A-FIG. 2K. Predicting therapy responses in small cell lung cancer patient derived xenografts using rag2^{null/null}; Il2rga−/− immunocompromised zebrafish. FIG. 2A-FIG. 2H: Cells of patient derived xenografts derived from small cell lung cancer patient, 1518 B3 p6 m58 (olaparib+temozlomide therapy sensitive) and 1528 2A5 p7 m10 (olaparib and temozolomide therapy resistant) were stained with cell lineage tracing dye, transplanted into rag2^{null/null}; Il2rga−/− immunocompromised zebrafish, imaged on 7 day post engrafted, oral gavaged with 50 ug/ml temozlomide and 25 ug/ml olaparib, and imaged again on 14 day post engraftment for therapy response. CSFE-stained cells from Patient 1518 B3 p6 m58 were sensitive to treatment and decreased CSFE intensity over time (FIG. 2A, FIG. 2I) and subsequently decreased cell number and proliferation when assessed on section using hematoxylin and eosin staining (FIG. 2B) and Ki67 immunohistochemistry staining (FIG. 2C, FIG. 2J). Cells also had increased apoptosis (FIG. 2D, FIG. 2K). In contrast, cells from therapy resistant patient 1528 2A5 p7 m10 did not respond to treatment, with retained engraftment after therapy (FIG. 2E, FIG. 2I), as well as no difference in cell count (FIG. 2F), proliferation (FIG. 2G, FIG. 2J) and apoptosis (FIG. 2H, FIG. 2K). Anova followed by Student's t-test with p<0.05 in panel I and p<0.001 in J,K (denoted by ***). The labeling on the left side of FIG. 2A also applies to FIG. 2B-FIG. 2D. The labeling on the left side of FIG. 2E also applies to FIG. 2F-FIG. 2H.

FIG. 3A-FIG. 3F. Using rag2^{null/null}; Il2rga−/− immunocompromised zebrafish to evaluate Car T-cell responses in vivo. FIG. 3A: Representative images of rag2^{null/null}; Il2rga−/− immunocompromised zebrafish engrafted with GFP-expressing U87 glioma cell line, intraperitoneally transplanted with either untransduced CD8+ T cells (top), non-specific CD19 Car T-cells (middle) or target-specific EGFRvIII Car T-cells, imaged before therapy on 7 days post transplant (dpt) and at end of experiment (21 dpt). FIG. 3B-FIG. 3D) Histological analysis (FIG. 3B), cell proliferation (FIG. 3C) and apoptosis (FIG. 3D) of engrafted U87 tumor sections from zebrafish subjected with either untransduced CD8+ T cells (top), non-specific CD19 Car T-cells (middle) or target-specific EGFRvIII Car T-cells. FIG. 3E: Quantification of U87 cell growth following injections of either untransduced T cell, non-specific CD19 Car T-cells or target-specific EGFRvIII Car T-cells. FIG. 3F: Quantification of JeKO-1 leukemia cell growth following injection of either untransduced T cell, non-specific EGFRvIII Car T-cells or target-specific CD19 Car T-cells. Fisher Exact Test (p=0.001, denoted by *** in FIG. 3E and FIG. 3F). The labeling on the left side of FIG. 3A also applies to FIG. 3B-FIG. 3D.

FIG. 4A-FIG. 4F. Using rag2^{null/null}; Il2rga−/− immunocompromised zebrafish to evaluate the efficacy of Bispecific T cell engagers (BiTES) in curbing tumor growth. FIG. 4A: Representative images of rag2^{null/null}; Il2rga−/− immunocompromised zebrafish engrafted with GFP-expressing OVCAR5 ovarian carcinoma cell line, intraperitoneally transplanted with CD8+ T cells and either EPCAM antibody (top), non-specific CD19 targeting BiTES, blinatumomab (middle) or target-specific EPCAM targeting BiTES, solitomab, imaged before therapy on 7 dpt and at end of experiment (21 dpt). FIG. 4B-FIG. 4D) Histological analysis (FIG. 4B), cell proliferation (FIG. 4C) and apoptosis (FIG. 4D) of engrafted OVCAR5 tumor sections from zebrafish subjected with CD8+ T cells and either EPCAM antibody (top), non-specific CD19 targeting BiTES, blinatumomab (middle) or target-specific EPCAM targeting BiTES, solitomab. FIG. 4E: Quantification of growth of OVCAR5 cells following different therapies. FIG. 4F: Quantification of growth of K562-CD19 (CD19) leukemia cells subjected to different therapies. Fisher Exact Test (p≤0.002, denoted by *** in panels FIG. 4E and FIG. 4F). The labeling on the left side of FIG. 4A also applies to FIG. 4B-FIG. 4D.

FIG. 5A-FIG. 5F. Using rag2^{null/null}; Il2rga−/− immunocompromised zebrafish to evaluate antibody-peptide epitope conjugates (APEC) efficiency. FIG. 5A: Representative images of rag2^{null/null}; Il2rga−/− immunocompromised zebrafish engrafted with GFP-expressing OVCAR5 ovarian carcinoma cell line, intraperitoneally transplanted with CMV primed T cells and either EPCAM antibody (top), non-specific EGFR targeting APEC (middle) or target-specific EPCAM targeting APEC, imaged before therapy at 7 dpt and at end of experiment (21 dpt). FIG. 5B-FIG. 5D: Histological analysis (FIG. 5B), cell proliferation (FIG. 5C) and apoptosis (FIG. 5D) of engrafted OVCAR5 tumor sections from zebrafish transplanted with CMV primed T cells and either EPCAM antibody (top), non-specific EGFR targeting APEC (middle) or target-specific EPCAM targeting APEC. (FIG. 5E): Quantification of OVCAR5 (EPCAM) cell growth following different therapies. (FIG. 5F): Quantification of MDA-MB-231 breast cancer cells (EGFR) subjected to different therapies. Fisher Exact Test (p≤0.001, denoted by *** in FIG. 5E and FIG. 5F). The labeling on the left side of FIG. 5A also applies to FIG. 5B-FIG. 5D.

FIG. 6A-FIG. 6E. Single cell resolution imaging of human cancer cells and responses to Car T-cell therapy in rag2^{null/null}; Il2rga−/− zebrafish. FIG. 6A: Representative confocal images of engrafted U87 cells before (6 dpt) and after (7, 11 and 14 dpt) transplantation of mCherry+human CD8+ T cells (top) or EGFRvIII Car T-cells (bottom). FIG. 6B: Quantification of total cell number in engrafted animals subjected to either control T-cells or EGFRvIII Car T-cells. FIG. 6C: Quantification of the total number of control T cells or EGFRvIII Car T-cells that had infiltrated into the engrafted U87 tumors within each image plane. FIG. 6D: 3D modeling of engrafted cells in 11 dpt animals subjected to either control CD8+ T-cells (left) or EGFRvIII Car T-cells (right). White arrowheads denote Car T-cells in close proximity to tumor cells. FIG. 6E: Quantification of distance from the tumor cells to the nearest control CD8+ T cell or EGFRvIII Car T-cells. Black bars represent mean distance. For each day (7, 11, or 14), data on left is for control and data on the right is for EGFRvIII. Asterisks denote statistical differences based on Student's T-test (*, p<0.05 and **, p<0.01).

FIG. 7A-FIG. 7E. In vivo quantification of BiTE-induced cell death using the zipGFP-Caspse 3 reporter in rag2^{null/null}; Il2rga−/− zebrafish. FIG. 7A: Representative confocal images of engrafted OVCAR5 cells transfected with zipGFP caspase 3 reporter, cells with activated Caspase 3 express both mCherry and GFP. Engrafted zebrafish are imaged before (6 dpt) and after (7, 11 and 14 dpt) transplantation of CSFE stained human CD8+ T cells with control EPCAM antibody (top) or solitomab (bottom). FIG. 7B, FIG. 7C: Quantification of total cell number (FIG. 7B) and cell apoptosis (FIG. 7C) in engrafted animals subjected to transplantation of CSFE stained human CD8+ T cells with either control EPCAM antibody or solitomab. FIG. 7D: 3D modeling of engrafted cells at 11 dpt in animals subjected to either control EPCAM antibody (left) or solitomab (right). White arrows denotes apoptotic cells. T cells are visibly in contact with apoptotic cells only in the solitomab treated group but not in animals treated with EPCAM antibody. FIG. 7E: Quantification of percentage of apoptotic cells in contact with T cells. Student's T-test comparisons (p<0.05 in FIG. 7B, FIG. 7C, and FIG. 7E).

FIG. 8A-FIG. 8E. Visualizing APEC therapy target specificity in rag2^{null/null}; Il2rga−/− zebrafish. FIG. 8A: Representative confocal image of zebrafish coinjected with GFP-expressing MDA-MB-231 breast cancer cells engineered to express EPCAM, and WT mCherry-expressing EPCAM-MDA-MB-231 cells. Engrafted zebrafish were imaged before (6 dpt) and after (7, 11 and 14 dpt) transplantation of CSFE stained human CMV primed T cells with control EPCAM antibody (top) or EPCAM targeting APEC (bottom). FIG. 8B-FIG. 8C: Quantification total cell number in engrafted animals subjected to either control EPCAM antibody (FIG. 8B) or EPCAM APEC (FIG. 8C). FIG. 8D: 3D modeling of engrafted cells at 11 dpt in animals subjected to either control EPCAM antibody (left) or EPCAM APEC (right). White arrows denote T cells in contact with EPCAM-expressing cancer cells. FIG. 8E: Quantification of percentage of tumor cells that is closest to transplanted T cells. Student's T-test comparison (p<0.05 in panel D, 14 dpt) and Fisher Exact Test (p<0.05 in panel FIG. 8E). Y axis is % of cells.

FIG. 9A-FIG. 9C. In vivo screen to identify the most effective APEC in killing ovarian carcinoma cells following engraftment into rag2^{null/null}; Il2rga−/− zebrafish. FIG. 9A: Schematic of in vivo screen using the OVCAR5 cell line expressing zipGFP Caspase 3 reporter and engraftment into the rag2^{null/null}; Il2rga−/− zebrafish. FIG. 9B: Representative confocal images of engrafted OVCAR5 cells that express the zipGFP Caspase 3 reporter and administered CMV primed T cells with either MMP7-APEC (top), TMPRSS4-APEC (middle) or uncleavable negative control APEC (bottom). FIG. 9C: Quantification of APEC induced cell apoptosis following APEC. Left arrow denotes the best APEC that kills ovarian cancer cells in vivo (MMP7(AVSRLRAY)) compared to control APEC (right arrow) or APEC with no in vivo efficacy (middle arrow).

FIG. 10A-FIG. 10J. Long-term effects of administering MMP7(AVSRLRAY) APEC on rag2^{null/null}; Il2rga−/− zebrafish engrafted with ovarian cancer cells. FIG. 10A-FIG. 10D: Representative images of rag2^{null/null}; Il2rga−/− zebrafish engrafted with OVCAR5 ovarian carcinoma cells, imaged before (7 dpt) and after (21 day) either administered control EPCAM antibody (top) or MMP7-APEC therapy (bottom) (FIG. 10A). Hematoxylin and Eosin staining (FIG. 10B), Ki67 (FIG. 10C) and TUNEL (FIG. 10D) immunohistochemistry staining of tumor sections from these animals. FIG. 10E: Quantification of relative growth rate in animals either administered control EPCAM antibody (black) or MMP7-APEC therapy. FIG. 10F-FIG. 10I: Representative images of rag2^{null/null}; Il2rga−/− zebrafish engrafted with MCAS ovarian carcinoma cells, imaged before (7 dpt) and after (21 day) either administered control EPCAM antibody (top) or MMP7-APEC therapy (bottom, FIG. 10F). Hematoxylin and Eosin staining (FIG. 10G), Ki67 (FIG. 10H) and TUNEL (FIG. 10I) immunohistochemistry staining of tumor sections from these animals. FIG. 10J: Quantification of relative growth rate in animals either administered control EPCAM antibody (black) or MMP7-APEC therapy. Fisher exact test comparisons shown in FIG. 10E and FIG. 10J. The labeling on the left side of FIG. 10A also applies to FIG. 10B-FIG. 10D.

FIG. 11A-FIG. 11F. Validating effect of MMP7(AVSRLRAY) APEC on killing ovarian cancer cells engrafted in NSG immunocompromised mice. FIG. 11A-FIG. 11B: NSG mice engrafted with luciferased OVCAR5 cells engrafted orthopedically and administered with either control EPCAM antibody (FIG. 11A) or MMP-7-APEC (FIG. 11B; top is day 0, bottom is day 24). FIG. 11C-FIG. 11D: Representative images of hematoxylin and eosin staining, Ki67 and TUNEL immunohistochemistry stainings on tumor sections from engrafted mice administered with either control EPCAM antibody (FIG. 11C) or MMP-7-APEC (FIG. 11D). FIG. 11E: Quantification of luciferase intensity in NSG mice engrafted with luciferased OVCAR5 cells engrafted orthopedically and administered with either control EPCAM antibody (black) or MMP-7-APEC. (F) Kaplan Meier survival analysis of NSG mice engrafted with luciferased OVCAR5 cells engrafted orthopedically and administered with either control EPCAM antibody (black) or MMP-7-APEC. Log-rank statistic (p<0.01 in FIG. 11E and FIG. 11F).

FIG. 12A-FIG. 12B: nk-lysin^{null/null} and il2rgb−/− immunocompromised zebrafish FIG. 12A: Schematic of 43 kb genome deletion in the nk-lysin gene locus, with boxes labeled “43% indel freq” indicating efficiency of CAS9/gRNA deletions in mosaic injections and subsequent generation of stable fish with nkl-locus deletion. The genomic sequences targeted by the CRISPR/Cas9 guide RNAs (top). Target sequences of guide RNA targeting the nk-lysin gene bottom). SEQ ID NOs: 5 (top left), 6 (top right), 7 (bottom left), and 8 (bottom right). FIG. 12B: Schematic of il2rgb gene mutation, with a 7 base pair frameshift deletion, creating a premature stop codon at the 33rd amino acid, with sequences under the line indicating genomic sequences targeted by TALEN arms. SEQ ID NOs:9 and 10, top to bottom, respectively.

### DETAILED DESCRIPTION

Immune compromised mice have been transformative in assessing the cellular functions of normal stem cell fractions and malignant cells in both mouse and human. For example, cell transplantation into immune compromised mice has been used extensively to identify stem and progenitor cells in various tissues including muscle [1-5], blood [6-12], skin [13], heart [14], and endodermal tissues including pancreatic beta-cells [15], hepatocytes, and, intestine [16, 17] and to assess regenerative capacity in a wide range of normal, aged, and diseased tissues. Cell engraftment into immune compromised mice is also a powerful experimental platform to uncover mechanistic insights into self-renewal, homing, migration, and regeneration in vivo.

To date, xenograft transplantation has utilized adoptive transfer of human cells into immune compromised mice including Nude (Foxn1-deficient) [18], NOD/SCID (NOD strain mice with DNA-dependent protein kinase, catalytic subunit-deficiency (Prkdc)) [19-21], and Rag-deficient strain animals [22, 23]. These strains lack fully functional T and/or B cells, but retain largely intact natural killer (NK)-cell function. For example, Non Obese Diabetic strain mice—commonly known as NOD mice—lack complement activity, have defects in myeloid development and antigen presentation, and have reduced NK cell activity. Nude mice have impaired thymic epithelial development resulting in disrupted T-lymphopoiesis. By contrast, Rag2- and Prkdc-deficient mice are unable to recombine T- and B-cell receptors, resulting in loss of functional lymphocytes. To obviate innate immune rejection mediated by NK cells, investigators have utilized Interleukin-2-gamma receptor (Il2rg)-deficient mice [24-26]. Il2rg heterodimerizes with a wide array of cytokine-specific interleukin receptors to orchestrate the cell signaling required for T and NK cell maturation. Creation of NOD/SCID/Il2rg−/− mice has resulted in nearly complete immune compromised animals and have facilitated robust adoptive transfer of both mouse and human cells into recipient animals [25, 26]. NK cell function can also be disrupted by inactivation of B2-microglobulin (B2m), perforin (Prf1), and janus kinase 3 (Jak3) [27] [27-30]. In total, a large number of immune compromised mouse models have been developed for use in cell transfer experiments.

Evolutionarily conserved pathways regulate immune competency. In fact, morpholino and gene inactivation studies in zebrafish has shown that jak3, rag1/2, and foxn1 regulate lymphocyte cell development [31-33], yet SCID phenotypes in adult fish and use of mutant zebrafish as recipients in cell transplantation have not yet been fully optimized. Creation of zebrafish deficient for rag2, prkdc, NK-lysin, il2rga, and/or il2rgb will likely provide powerful models for cell transplantation of zebrafish, mouse, and human cells—facilitating the next generation of low-cost, high throughput cell transplantation models.

Zebrafish have many attributes that represent clear advantages over more commonly used vertebrate models, including 1) fecundity: each female can produce 100-200 eggs per week; 2) small size: thousands of animals can be reared in a relatively small space; 3) reduced cost: mouse per diems range from $0.20-$1.00/day depending on cage size, while fish per diems are <$0.01/day, 4) optical clarity: engraftment of normal and malignant cells can be easily visualized by fluorescent labeling and direct visualization of engrafted cells can be further enhanced by zebrafish lines that lack pigment—aptly called Casper [34]; 5) wide temperature range: zebrafish can be reared at 18° C.-37° C., the latter mimicking temperatures seen in mouse and human, 6) high-throughput cell transplantation: 350+ adult syngeneic zebrafish can be transplanted intraperitoneally or retro-orbitally with fluorescently-labeled cancer cells by one investigator in a single day, facilitating large scale experimentation [35-39]. To date, the major hurdle for use of zebrafish as a xenograft transplant model is lack of immune-compromised zebrafish strains.

Blood development and function are highly conserved between zebrafish, mouse, and human [40-44]. Zebrafish have a well-developed acquired and innate immune system that develops during the first weeks of life that can detect and kill foreign cells. Zebrafish have T, B, and NK cells as well as myeloid, erythroid, and precursor cell populations. Capitalizing on the short window of immune tolerance in early larval development, investigators have utilized cell transplantation of human cancer cells into 2-day-old zebrafish [45-50]. However, animals eventually develop immune responses and kill engrafted cells—preventing analysis of animals after 7 days of life. Moreover, only 20-200 cells can be implanted into larval fish due to their small size. The next generation of cell transplantation utilized transient ablation of the immune system by gamma-irradiation, allowing robust engraftment of tumor and hematopoietic cells for up to 20 days post-transplantation where in excess of 1×10⁶ cells can be implanted per fish [39, 51]. However, the recipient immune system recovers by 30 days post-irradiation and attacks the graft leading to rejection. Recent experiments have shown robust blood cell engraftment of zebrafish blood cells into myb-deficient fish that lack fully functional blood stem cells [52]. Although powerful for study of blood development, myb mutant fish die in early adulthood and are not amenable to adoptive transfer of other cell types nor have they been used in xenograft assays. Work from the Klemke laboratory has shown that immune suppression by dexamethasone permits engraftment of a wide variety of human tumors to time points exceeding 30 days [53], strongly arguing that human cancer can survive and grow in adult fish. However, these protocols result in only partial immune suppression through impairment of lymphocyte function, require constant dosing of drugs, and would not be useful for assessment of hematopoietic and leukemia cell engraftment.

Finally, syngeneic strains of zebrafish facilitate robust and large-scale engraftment studies using fluorescent-labeled zebrafish cells [35-38]. These models require that donor and recipients are both in the same strain and engraftment of mammalian cells into these models is not possible as they are not immune compromised. To date, immune compromised zebrafish have yet to be used for universal cell transplantation of either zebrafish or mammalian cells.

Genome engineering and creation of mutant zebrafish Robust methods to induce targeted gene disruption in zebrafish using zinc finger nucleases (ZFNs) and Transcription Activator-Like Effector Nucleases (TALENs) have been developed [54-65].

ZFNs consist of an engineered array of zinc fingers fused to the non-specific FokI nuclease domain and function as dimers to introduce targeted DNA double-strand breaks (DSBs). Each zinc finger binds to approximately three base pairs (bps) of DNA and a ZFN monomer commonly utilizes three to six zinc finger motifs to bind 9-18 bp target DNA. See, e.g., Miller et al., 1985, EMBO J., 4:1609; Berg, 1988, Proc. Natl. Acad. Sci. USA, 85:99; Lee et al., 1989, Science. 245:635; and Klug, 1993, Gene, 135:83; Rebar et al., 1994, Science, 263:671; Choo et al., 1994 Proc. Natl. Acad. Sci. USA, 91:11163; Jamieson et al., 1994, Biochemistry 33:5689; Wu et al., 1995 Proc. Natl. Acad. Sci. USA, 92: 344; Segal et al., 2003, Biochemistry, 42:2137-48; Beerli et al., 2002, Nat. Biotechnol., 20:135-141; Mandell et al., 2006, Nucleic Acids Res., 34:W516-523; Carroll et al., 2006, Nat. Protoc. 1:1329-41; Liu et al., 2002, J. Biol. Chem., 277:3850-56; Bae et al., 2003, Nat. Biotechnol., 21:275-280; Wright et al., 2006, Nat. Protoc., 1:1637-52. Combinatorial selection-based methods that identify zinc finger arrays from randomized libraries have been shown to have higher success rates than modular assembly (Maeder et al., 2008, Mol. Cell, 31:294-301; Joung et al., 2010, Nat. Methods, 7:91-92; Isalan et al., 2001, Nat. Biotechnol., 19:656-660). In preferred embodiments, the zinc finger arrays are described in, or are generated as described in, WO 2011/017293 and WO 2004/099366. Additional suitable zinc finger DBDs are described in U.S. Pat. Nos. 6,511,808, 6,013,453, 6,007,988, and 6,503,717 and U.S. patent application 2002/0160940.

By contrast, TALENs bind to DNA through a highly conserved 34 amino acid transcription activator-like effector (TALE) repeat domain found in the plant pathogen Xanthomonas. Each TALE repeat domain binds to a single bp of DNA with specificity determined by two amino acids—known as the repeat variable di-residues (RVDs). TALEs can be joined together into extended arrays to create proteins that bind longer stretches of DNA sequence. TALE repeats are fused to the FokI nuclease domain and cleave DNA as a dimer. Methods for creating and using TALENs are well known in the art, see, e.g., Reyon et al., Nature Biotechnology 30, 460-465 (2012); as well as the methods described in Bogdanove & Voytas, Science 333, 1843-1846 (2011); Bogdanove et al., Curr Opin Plant Biol 13, 394-401 (2010); Scholze & Boch, J. Curr Opin Microbiol (2011); Boch et al., Science 326, 1509-1512 (2009); Moscou & Bogdanove, Science 326, 1501 (2009); Miller et al., Nat Biotechnol 29, 143-148 (2011); Morbitzer et al., T. Proc Natl Acad Sci USA 107, 21617-21622 (2010); Morbitzer et al., Nucleic Acids Res 39, 5790-5799 (2011); Zhang et al., Nat Biotechnol 29, 149-153 (2011); Geissler et al., PLoS ONE 6, e19509 (2011); Weber et al., PLoS ONE 6, e19722 (2011); Christian et al., Genetics 186, 757-761 (2010); Li et al., Nucleic Acids Res 39, 359-372 (2011); Mahfouz et al., Proc Natl Acad Sci USA 108, 2623-2628 (2011); Mussolino et al., Nucleic Acids Res (2011); Li et al., Nucleic Acids Res 39, 6315-6325 (2011); Cermak et al., Nucleic Acids Res 39, e82 (2011); Wood et al., Science 333, 307 (2011); Hockemeye et al. Nat Biotechnol 29, 731-734 (2011); Tesson et al., Nat Biotechnol 29, 695-696 (2011); Sander et al., Nat Biotechnol 29, 697-698 (2011); Huang et al., Nat Biotechnol 29, 699-700 (2011); and Zhang et al., Nat Biotechnol 29, 149-153 (2011); all of which are incorporated herein by reference in their entirety.

The DSBs induced by either ZFNs or TALENs are repaired by non-homologous end joining (NHEJ)—an error-prone process that results in the creation of insertion or deletion mutations (indels) that can shift the frame and lead to premature translation termination. Using targeted genomic engineering approaches, DNA mutations have been successfully targeted within somatic cells of zebrafish [for a review see 84]; some of these have produced heritable loss-of-function mutations; see, e.g., [54, 56-67], all of which are incorporated herein by reference in their entirety.

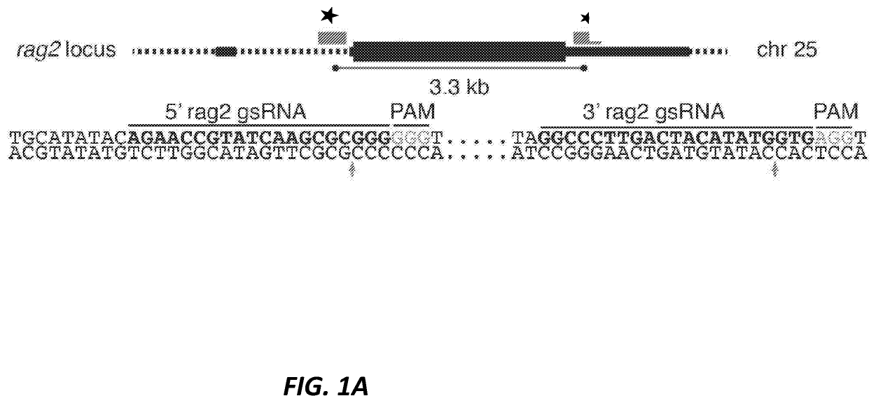

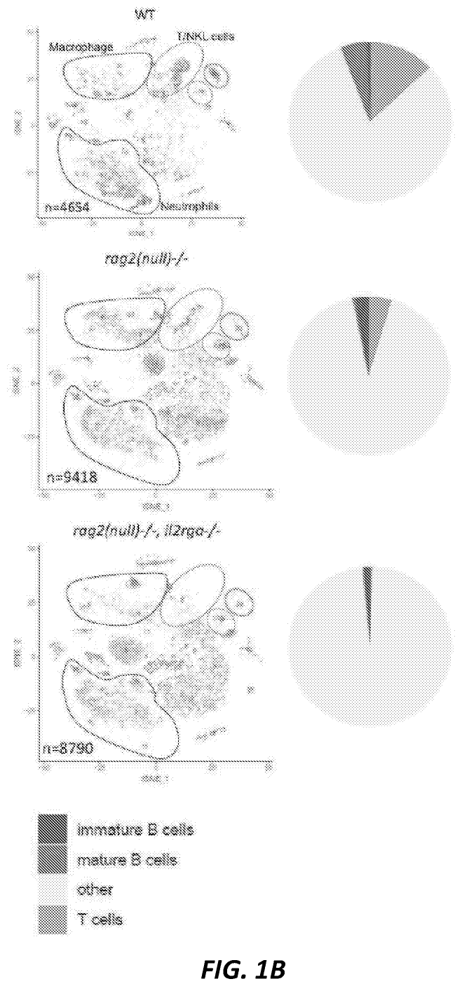

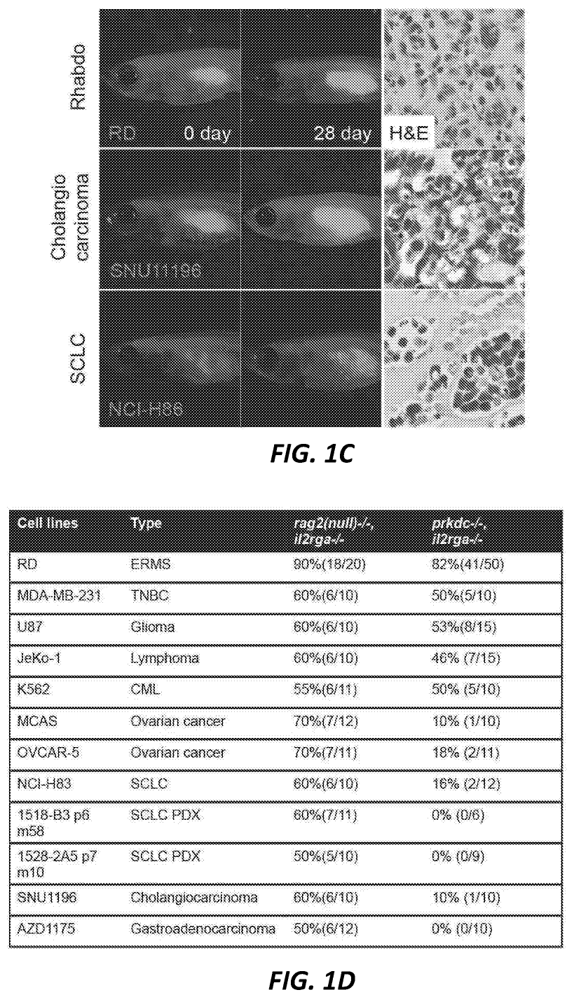

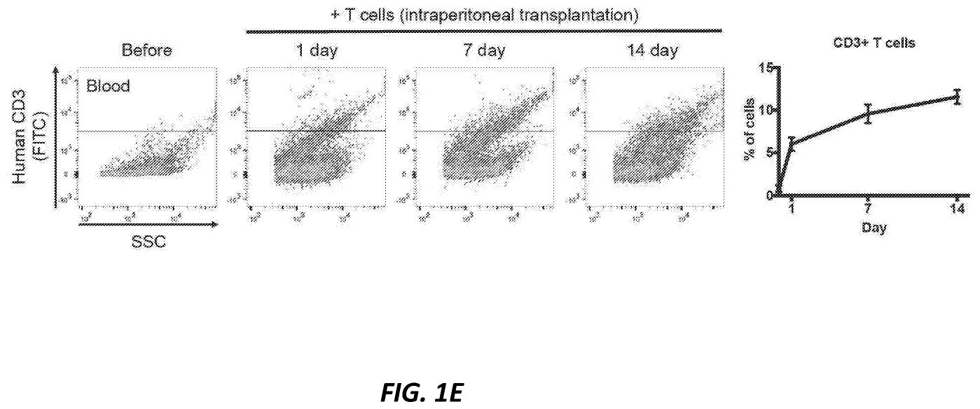

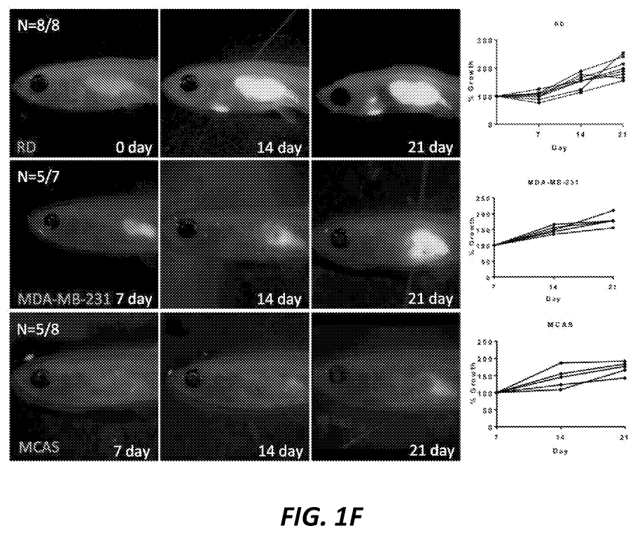

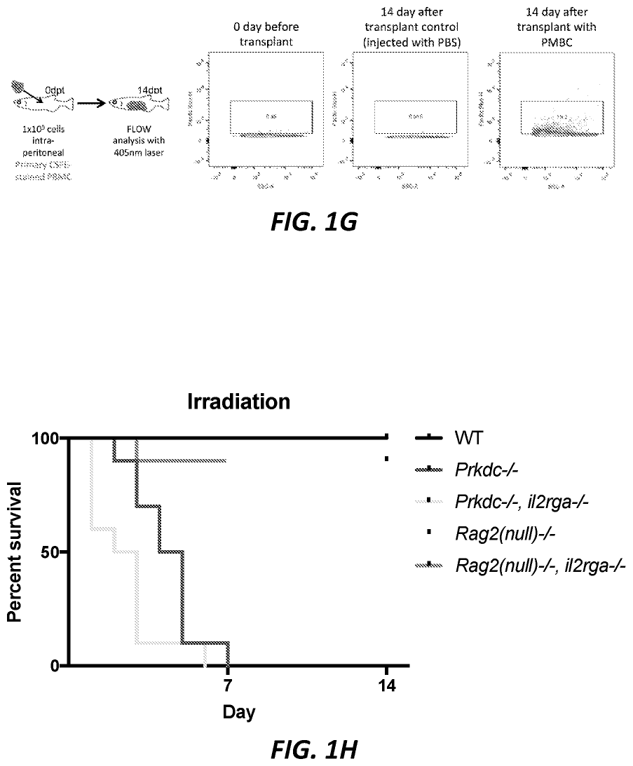

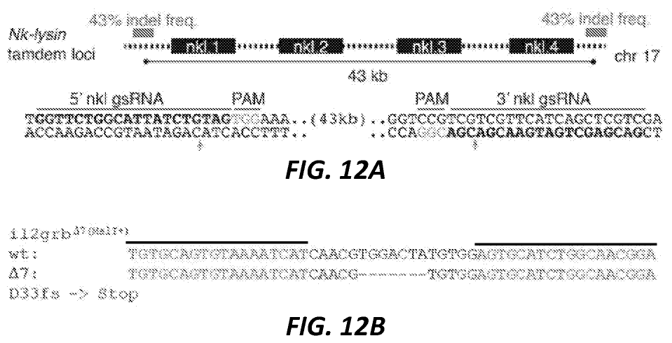

The clustered regularly interspaced short palindromic repeats (CRISPR)/CRISPR-associated protein 9 (Cas9) system may also be used to genetically modify the zebrafish described herein. Methods of using CRISPR/Cas9 for genome editing in zebrafish are known in the art (see, e.g., Cornet et al., Frontiers in Pharmacology, 2018, 9(703)). Genome editing with CRISPR/Cas9 of the present disclosure include at least two components adapted from naturally occurring CRISPR systems: a gRNA (see, e.g., FIG. 1A and FIG. 12A) and an RNA-guided nuclease (e.g., Cas9). These two components form a complex that is capable of associating with a specific nucleic acid sequence in a cell and editing the DNA in or around that nucleic acid sequence, for example by making one or more of a single-strand break (an SSB or nick), a double-strand break (a DSB) and/or a base substitution. See, e.g., WO2018/026976 for a full description of genome editing systems.

Fish

A wide variety of fish species, including teleosts, can be utilized to generate the immune-compromised genetically-modified fish disclosed herein. Suitable teleosts include, e.g., zebrafish (Danio rerio); medaka (Oryzias latipes); mummichog (Fundulus heteroclitus); killifish (Genus Fundulus); catfish (Genus Ictalurus), such as channel catfish; carp (Genus Cyprinus), such as common carp; puffer fish (Tetraodontidae); and trout or salmon (such as Genus Salvelinus, Salmo, and Oncorhynchus). In some embodiments, the fish models are transparent or translucent in one or more of the following stages: the embryonic, larval, or adult stage.

Zebrafish offer important advantages over other fish models. The genetic makeup of zebrafish is closely related to other vertebrates, including human and mouse, thus zebrafish serves as an excellent model for the study of vertebrate development and human diseases. Zebrafish share with mammals major lymphoid organs such as thymus and gut-associated lymphoid tissues. Although zebrafish do not possess lymph nodes or bone marrow, the species maintains a major hematopoietic activity in the kidney. Various zebrafish strains can be used to generate the immune-compromised genetically-modified fish. Suitable zebrafish strains include the wild-type strains such as AB, Tübingen, AB/Tübingen, Sanger AB Tübingen, SJD, SJA, WIK strains, and the pigmentation mutant strains such as golden, albino, rose, panther, leopard, jaguar, puma, bonaparte, cezanne, chagall, dali, duchamp, picasso, seurat, sparse, shady, oberon, opallus, nacre, roy, and (preferably) Casper strains.

Adult wild-type zebrafish have three classes of pigment cells arranged in alternating stripes: black melanophores, reflective iridophores, and yellow xanthophores. The pigmentation mutant strains harbor one or more mutations in the genes that play important roles in the development of melanophores, iridophores, or xanthophores. For example, the golden mutant zebrafish harbors a point mutation in the slc24a5 gene and has diminished number, size, and density of melanosomes (Lamason et al., Science 310(5755):1782-6, 2005). Similarly, the albino mutants also have a mutation in the slc24a5 gene and show very light body pigmentation (see, e.g., Lamason et al., Science. 310. (2005): 1782-86). The rose mutants harbor a mutation in the endothelin receptor b1 (ednrb1) gene and have fewer numbers of melanocytes and iridophores during pigment pattern metamorphosis and exhibit disrupted pattern of the melanocytes (Parichy et al., Dev. Biol. 227(2):294-306, 2000).

Several zebrafish mutant strains completely lack one or more classes of pigment cells. The panther mutants complete lack xanthophores and have fewer melanophores due to a mutated fms (M-CSF receptor) gene (Parichy et al., Development 127: 3031-3044, 2000). The nacre mutant of zebrafish completely lacks melanocytes due to a mutation in the mitfa gene (Lister et al., Development 126:3757-3767, 1999). The roy orbison (roy) zebrafish is a spontaneous mutant with unknown mutations, which cause a complete lack of iridophores, sparse melanocytes, and a translucency of the skin. The roy mitfa^{-/-} double mutant zebrafish designated “Casper” shows a complete loss of all melanocytes and iridophores [34]. The body of Casper fish is almost entirely transparent during both embryogenesis and adulthood, and the internal organs, including the heart, aorta, intestinal tube, liver, and gallbladder, can be seen using standard stereomicroscopy. In female Casper fish, individual eggs are also observable. The Casper mutant zebrafish is entirely viable, with incrossed adults producing large numbers of viable offspring at expected mendelian ratios and no heterozygous phenotype [34]. By utilizing the optically clear Casper strain fish, engraftments in the fish can be directly visualized through the use of reporter dyes or proteins by a variety of detection techniques, e.g., light microscopy, fluorescence microscopy, colorimetry, chemiluminescence, digital imaging, microplate reader techniques, and in situ hybridization. Therefore, the Casper strain fish is particularly suitable for the experiments disclosed herein.

In some aspects, the fish described herein are tolerant to irradiation (e.g., survive for at least 14 days after exposure to 20 gy gamma-irradiation irradiation).

Immune-Related Genes

The genetically modified fish described herein have germline mutations in one or more immune-related genes that render their immune systems less active than wild type. In some embodiments, their immune systems are inactive. In some embodiments, the fish lack one or more of T, B, and/or NK cells, or have inactive T, B, and/or NK cells. The immune-related genes that are inactivated in the fish described herein include one or more of rag2, prkdc, NK-lysin, il2rga, and il2rgb. In some embodiments, the mutations are as shown in FIG. 1A, FIG. 12A, or FIG. 12B. Methods of producing mutant zebrafish are also described in International Patent Application Publication No. WO 2015/006455.

Rag2

The recombination-activating gene 2 (rag2) encodes a protein that is involved in the initiation of V(D)J recombination during B and T cell development. The protein RAG-2 forms a complex with RAG-1, and this complex cleaves DNA at conserved recombination signal sequences and forms double-strand breaks in DNA. Both RAG-1 and RAG-2 are essential to the generation of mature B and T lymphocytes. The rag1 mutant zebrafish are able to reach adulthood and are fertile [33]. The sequences of mRNA, genomic DNA, and protein of zebrafish rag2 are known in the art and their GenBank Reference Numbers are listed in the table below.

| rag2 | GenBank Reference No. |
|---|---|
| mRNA | Accession: NM_131385.2 GI: 119943149 |
| Genomic DNA | Accession: NC_007136.5 GI: 312144705 |
| Protein | Accession: NP_571460.2 GI: 119943150 |

The rag2 deletion is homozygous, i.e., results in mutant zebrafish that lack the rag2 gene. The resulting rag2 fish are amorphic. See, e.g., FIG. 1A. The rag2 deletion is generated by deleting ˜3.3 kb of the rag2 gene through CRISPR/Cas9 genome editing. The target sequences of the guide RNA targeting the rag2 gene are depicted in FIG. 1A.

Il2rga/Il2rgb

The interleukin-2 receptor gamma chain (Il2rg) is an important signaling component of many interleukin (IL) receptors, including receptors for IL-2, IL-4, IL-7, IL-9, IL-15, and IL-21, and is thus referred to as the common gamma chain. Deletion of Il2rg in the NOD/SCID mice results in lack of functional lymphocytes and nature killer (NK) cells. The sequences of mRNA, genomic DNA, and protein of zebrafish Il2rga and Il2rgb are known in the art and their GenBank Reference Numbers are listed in the table below.

| Il2rga | GenBank Reference No. |
|---|---|
| mRNA | Accession: NM_001128271.1 GI: 190194257 |
| Genomic DNA | Accession: NC_007121.5 GI: 312144720 |
| Protein | Accession: NP_001121743.1 GI:190194258 |

| Il2rgb | GenBank Reference No. |
|---|---|
| mRNA | Accession: NM_001123050.1 GI: 176866336 |
| Genomic DNA | Accession: NC_007125.5 GI: 312144716 |
| Protein | Accession: NP_001116522.1 GI:176866337 |

Prkdc

The DNA-dependent protein kinase catalytic subunit (prkdc) gene encodes the catalytic subunit of the DNA-dependent protein kinase (DNA-PK). DNA-PK functions with the Ku70/Ku80 heterodimer protein in DNA double strand break repair and recombination. Defective DNA-PK activity is linked to V(D)J recombination defects and DNA repair defects associated with the murine SCID mutation [20]. The sequences of mRNA, genomic DNA, and protein of zebrafish prkdc are known in the art and their Genbank Reference Numbers are listed in the table below.

| prkdc | GenBank Reference No. |
|---|---|
| mRNA | Accession: XM_001919553.2 GI: 326669530 |
| Genomic DNA | Accession: NC_007118.5 GI: 312144723 |
| Protein | Accession: XP_001919588.2 GI:326669531 |

NK-Lysin

The family of NK-lysin genes encode antimicrobial proteins produced by cytotoxic T lymphocytes and natural killer cells. In zebrafish there are four different NK-lysin genes: nkla, nklb, nklc and nkld. NK-lysins are stored in cytoplasmic granules and released upon target cell recognition via MHC class I to induce apoptosis. The sequences of mRNA, genomic DNA, and protein of zebrafish NK-lysins are known in the art and their GenBank Reference Numbers are listed in the table below.

| nkla | GenBank Reference No. |
|---|---|
| mRNA | Accession: NM_001311794.1 GI: 909122005 |
| Genomic DNA | Accession: NC_007128.7 GI: 1196813936 |
| Protein | Accession: NP_001298721.1 GI: 914614922 |

| nklb | GenBank Reference No. |
|---|---|
| mRNA | Accession: NM_001311792 GI: 909122008 |
| Genomic DNA | Accession: NC_007128.7 GI: 1196813936 |
| Protein | Accession: NP_001298721.1 GI: 914615414 |

| nklc | GenBank Reference No. |
|---|---|
| mRNA | Accession: NM_001311793 GI: 909122011 |
| Genomic DNA | Accession: NC_007128.7 GI: 1196813936 |
| Protein | Accession: NP_001298722.1 GI: 914615960 |

| nkld | GenBank Reference No. |
|---|---|
| mRNA | Accession: NM_212741 GI: 909122014 |
| Genomic DNA | Accession: NC_007128.7 GI: 1196813936 |
| Protein | Accession: NP_997906.1 GI: 47086559 |

In some aspects, the fish described herein have combinations of these mutations, e.g., rag2, prkdc, NK-lysin, il2rga, and il2rgb. In some aspects, the fish described herein have mutations in rag2 and il2rga. In some aspects, the fish described herein have mutations in prkdc, NK-lysin, il2rga, and il2rgb.

In some aspects, the fish described herein are amorphic for rag2 and have the inactivating point mutationY92fs in the il2rga gene (referred to as rag2^{null/null}; il2rga−/−). See FIG. 1A for a description of how to generate the rag2 genotype and the methods in the Examples section for how to generate the rag2^{null/null}; il2rga−/− zebrafish.

In some aspects, the fish described herein are NK-lysin^{null/null} (see Example 2 below). See FIG. 12A for a description of how to generate the NK-lysin genotype and the methods in the Examples section for how to generate the NK-lysin^{null/null} zebrafish.

In some aspects, the fish described herein are il2rgb^{D33fs}−/− (see Example 2 below). See FIG. 12B for a description of how to generate the il2rgb^{D33fs}−/− genotype and the methods in the Examples section for how to generate the il2rgb^{D33fs}−/− zebrafish.

In some aspects, the fish described herein are prkdc^{D3612fs}−/−; NK-lysin^{null/null}; il2rga^{Y91fs}−/−; il2rgb^{D33fs}−/−. See Example 2 below and the methods in the Examples section for how to generate the prkdc^{D3612fs}−/−; NK-lysin^{null/null}; il2rga^{Y91fs}−/−; il2rgb^{D33fs}−/− fish.

The following table provides an additional list of zebrafish genes targeted for DNA deletion of functional domains or the entire coding locus:

| | | | |
|---|---|---|---|
| Rag1 | Il2rga | MCM4 | Cd3 chains |
| Rag2 | Il2rgb | Blm | Cd4 |
| Lck | Il7 | Perforins | Cd8 |
| Ikaros | Il7r | ADA | Cd25 |
| Prkdc | Pax5 | Dkc1 | CD40L |
| Jak1 | Ebf1 | Ikzf1 | Artemis |
| Jak2 | Nk-lysin | Mthfda | Ak2 |
| Jak3 | B2-microglobulin | Itgb2 | Pnp |
| Tox | MHC class II | Xiap | Cd45 |
| Tox3 | Cd40 | Plcg2 | |
| Tox4 | | | |

The fish described herein can also have combinations of these mutations.

Methods of Use

The fish described herein can be used, e.g., in stem cell biology, regenerative medicine, and cancer research. Fish facilitate large-scale transplantation experiments at greatly reduced costs to investigators. For example, 350+ adult zebrafish transplant experiments can be performed daily by a single investigator [35-38, 68]. Fish can also be engrafted at early stages of development beginning from the one cell stage of life on into adulthood. Such experiments further enhance the scale of experimentation due to reduction in animal size of larval fish permitting raising animals in 48-well formats in lowered amounts of water. Moreover, fish, e.g., zebrafish, can be housed and maintained at very low cost. In embodiments utilizing optically clear fish, e.g., the Casper strain zebrafish, engraftment can be directly visualized, e.g., through the use of fluorescent reporter dyes and proteins. Primary engrafted human and mouse tumors can often be directly visualized as masses, especially in the translucent Casper strains of fish given that internal zebrafish organs can be seen, facilitating tracking of human and mouse cells directly without dye or transgene labeling.

As described herein, the ability to create targeted gene mutations in zebrafish using ZFNs and TALENs, e.g., as described in [58, 64], or CRISPR/Cas9 e.g., as described in Cornet et al. (see above), can be used to effectively create zebrafish gene modifications that mimic mutations found in human and mouse SCID. Zebrafish can also be raised at 37° C. mimicking the temperatures seen in mouse and human, thus immune compromised fish will likely provide new experimental models for adoptive transfer of human and mouse cells to assess stem cell function and regenerative capacity. The use of large numbers of engrafted animals along with chemical screening approaches will likely revolutionize the types and scale of experiments that can be completed to assess mouse and human cellular functions—identifying the next generation of drugs to treat a variety of developmental, age-related, and cancer diseases.

The use of zebrafish for xenograft cell transplantation to assess stem cell phenotypes, regenerative capacity, and malignancy has lagged behind mouse models due in large part to the lack of immune compromised zebrafish. As described herein, ZFN, TALEN, and CRISPR/Cas9 technologies have been used for targeted gene inactivation in zebrafish. The present inventors have created TALENS and ZFNs that target endogenous genes for immune-related genes (e.g., il2rga, il2rgb, prkdc). In the case of TALEN/ZFN targeting, the mutations are engineered within the protein to create full-loss-of-function alleles. The present inventors have also created CRISPR/Cas9 guide RNAs that delete immune-related genes from the zebrafish genome (e.g., rag2, nk-lysin). A detailed description of these methods is provided in the Examples section below. The development of immune compromised zebrafish defective in these genes will facilitate large-scale transplantation experiments and robust methods for assessing cellular function, e.g., the cellular functions of normal stem cells and malignant cells.

The immune compromised genetically-modified fish disclosed herein can be used as transplant recipients to assess stem cell phenotypes, regenerative capacity and malignancy. In some embodiments, the stem cells are isolated from a donor zebrafish under sterile conditions. The donor fish can be treated with antibiotics and then euthanized, and the skin of the fish can be removed. The deskinned donor fish can then be briefly rinsed in bleach and homogenized, and cells of the donor fish can be purified, e.g., by Ficoll gradient, to eliminate bacteria and fungus. The cells of interest can be isolated and injected into the immune compromised genetically-modified fish disclosed herein. Transplanted fish can be examined using a variety of detection techniques, e.g., light microscopy, fluorescence microscopy, colorimetry, chemiluminescence, digital imaging, microplate reader techniques, and in situ hybridization. In some instances, the transplanted fish can be imaged at single cell resolution. Some embodiments utilize zebrafish to zebrafish transplantations that can be used, e.g., to assess regenerative capacity of muscle, blood, liver, kidney cells, pancreas (including beta-cells), skin, retinal cells, germ cells, and other regenerative tissues. Moreover, immune compromised fish can be used for cross-species engraftment of cells, e.g., normal cells, from other fish species, frogs, mouse, and human as well as cancer. In all instances, genetic and chemical approaches can be used to assess effects on regeneration and tumor growth—providing rapid methods to identify critical pathways that drive regeneration and cancer growth. In all instances, genetic and chemical approaches can be used to assess immune cell infiltration and tumor:immune cell interactions, location, and distances in vivo in the transplanted fish (e.g., at a single cell resolution).

Regeneration of the immune system can be studied, e.g., using fluorescent protein labeled hematopoietic stem cells (HSC). Since the kidney marrow is the site of hematopoiesis in zebrafish and contains the HSC cells, GFP-expressing kidney marrow cells can be isolated, e.g., from adult ubiquitin-GFP transgenic (Mosimann et al., Development. 2011 January; 138(1):169-77) and/or blood-specific promoter-GFP transgenic (Ellett et al., Blood Jan. 27, 2011 vol. 117 no. 4 e49-e56; Lam et al., Blood. 2009 Feb. 5; 113(6):1241-9) zebrafish kidneys and used for transplantation. Briefly, the donor zebrafish can be anesthetized and the kidneys can be dissected out under sterile conditions and placed into ice-cold sterile PBS buffer containing 5% fetal calf serum. Whole kidney marrow cell suspensions can be generated by aspiration followed by passing through a 40-μm nylon mesh filter. DnaseI and heparin can be added to lessen aggregation. The transplant recipient fish can be irradiated several days prior to transplant. Cells, e.g., approximately 1.5×10⁶ whole kidney marrow cells, e.g., in 5 ul volume, can be transplanted into each anesthetized recipient fish by retro-orbital injection as described before (Pugach et al., J Vis Exp 34:1645, 2009). The transplanted fish can be examined weekly under an inverted fluorescent microscope to monitor regenerative capacity of HSCs based on the distribution of GFP-expressing cells in the thymus, kidney, spleen, and other organs. Imaging of the transplanted fish can be captured using a digital camera. Fish can be sacrificed and GFP-expressing cells can be isolated from the kidney marrow, thymus, and spleen, and the cell lineages can be analyzed by fluorescence-activated cell sorting (FACS) and Wright-Giemsa/May-Grunwald staining. Successful engraftment can be defined by long-term and sustained GFP-positivity in all blood cell lineages. Loss of GFP-expressing cells in the blood denotes rejection, indicating the recipient fish is not immune compromised.

Regenerative capacity of muscle stem cells can be studied, e.g., using fluorescent protein labeled muscle stem cells. For example, muscle cells can be isolated from adult alpha-actin-RFP transgenic zebrafish utilizing the sterile techniques described above. A small amount of a toxin, e.g., snake venom (cardiotoxin) can be injected into the dorsal musculature of the genetically-modified recipient fish to damage the muscle fibers and trigger regeneration. Alternatively, muscle cell engraftment can be performed without pre-injury. Approximately 5×10⁴ RFP-expressing muscle cells, including muscle stem cells, can be injected into the dorsal musculature, at the same location as any pre-injury. The transplanted fish can be examined by fluorescent stereomicroscope to monitor the regenerative capacity of the muscle stem cells based on the distribution of RFP-expressing cells at the dorsal musculature of the fish.

In some embodiments, the genetically-modified fish disclosed herein can be used in cancer research. For example, GFP or RFP expressing mammalian tumor cells can be transplanted into the immune compromised genetically-modified zebrafish and tumor development can be directly visualized. Exemplary mammalian tumor cells include various sarcoma, carcinoma, adenocarcinoma, melanoma, and leukemia cell lines. About 1×10⁶ sterile tumor cells in 10 ul can be implanted into the peritoneal cavity of a recipient genetically-modified fish. Transplanted fish can be raised at 37° C. under semi-sterile conditions and can be examined, e.g., by fluorescent stereomicroscope, to monitor tumor growth in vivo. Tumors also need not be fluorescently tagged if they are engrafted into optically clear, Casper strain fish where tumors can be directly visualized.

The genetically-modified fish transplanted with mammalian tumor cells are useful to screen for therapeutic compounds that modulate tumor formation. The genetically-modified fish transplanted with tumor cells can be exposed to a test compound or a control substance. Tumor growth in the genetically-modified fish exposed to the test compound can be compared with the tumor growth in the genetically-modified fish exposed to the control substance. If a test compound suppresses or decreases tumor growth in the fish, it is identified as a candidate therapeutic compound; optionally the compound is selected and further assays can be conducted using the selected compound. The test compounds can be administered to the genetically-modified fish directly by microinjection, or oral gavage, or added to the water holding the genetically-modified fish, with the fish taking up the compound through their skin, gills, and gut. Thus a method of identifying a candidate therapeutic compound for the treatment of a mammalian tumor is provided by the present disclosure. The method includes transplanting cells from a mammalian tumor into the genetically-modified fish disclosed herein; contacting the fish with a test compound; evaluating the growth of the mammalian tumor cells in the presence of the test compound; comparing the level of growth of the mammalian tumor cells in the presence of the test compound to a reference level; and identifying a compound that decreases the level growth of the mammalian tumor cells as a candidate therapeutic compound. In some embodiments, the mammalian tumor cells are derived from a subject with cancer. In this way the fish can be used as a model to assess patient tumor responses to combined known therapies and for stratification into clinical trials—identifying tumors that best respond to therapies for the treatment of patients. In some embodiments, the identified candidate therapeutic compound is administered to the subject with cancer. Human and mouse primary cancers and cancer cell lines can be engrafted into immune compromised fish lines throughout development and into adult stages. In some instances, the test compound is a FDA-approved drug, a chimeric antigen receptor (CAR), a CAR-T cell, a bispecific T cell engager (BiTE), or an antibody peptide epitope circuit (APEC) therapy. In some instances, the method further comprises administering a T cell. In some instances, this method will use combination therapies including complexing the above single therapies together. In some instances, the method further comprises administering a T cell and performing single cell imaging. Single cell imaging may be used to monitor the infiltration of T cells into a tumor upon treatment with a test compound.

In some embodiments, the genetically-modified fish, e.g., transplant recipient fish, can be raised under sterile condition, optionally in the presence of one or more antibiotics and antifungals, e.g., Tetracycline, penicillin, fungazone, Cipro (Ciprofloxacin), bacitracin, and/or gentamycin.

In some embodiments, mammalian cells are transplanted into the fish, and before or after the transplant procedure the temperature of the fish container can be increased slowly, e.g., 0.75° C. daily, from 26.5° C. to 37° C. For example, intended transplant recipients can be acclimated to 37° C. for several days prior to engraftment with human or mouse cells.

The fish described herein, e.g., the zebrafish lines, can also be used to evaluate totipotency of stem cells, e.g., human, mouse, and other mammalian ES, iPS, and pluripotent cells. In these examples, investigators can engraft ES or modified cells into the fish and assess teratoma formation—a surrogate for pluripotency. The fish can be contacted with various test compounds, e.g., to discover drug combinations or genetic manipulations that alter cell fate. Teratoma assays are the gold standard for potency. The effects of the test compounds can also be evaluated. See, e.g., Science. 2003 Feb. 7; 299(5608):887-90.

For example, in some embodiments endogenous tissue-restricted, pluripotent stem cells (e.g., embryonic stem cells), or induced-pluripotent stem cells (iPSC), which can be, e.g., isolated from culture or in vivo, are purified and microinjected into fish. The stem cells can be placed into various locations in the fish including but not limited to the peritoneum, the vessels of the eye, the muscle, and various visceral organs. Drugs or genetic modifications (e.g., siRNA, antisense, or transgenes), e.g., drugs or genes that modulate specific pathways, can be delivered to the fish, and assessed for functional effects on growth or differentiation of the stem cells and their progeny using standard techniques including visualization of cells in vivo, sectioning and staining, etc. In some embodiments, the stem cells are labeled in some way, e.g., they express a fluorescent protein, so that the stem cells and their progeny are readily detectable.

### EXAMPLES

The invention is further described in the following examples, which do not limit the scope of the invention described in the claims.

### Example 1. Zebrafish Deletion and Compound Mutants for Use in Allogeneic and Xenograft Cell Transplantation

A New Rag2^{null/null}, il2rga^{-/-} Immunocompromised Zebrafish that can Robustly Engraft Human Cells

Adult zebrafish have many attributes that make them an ideal cell transplantation model, including high fecundity, low cost, optical clarity and the ability to perform high-throughput drug and tumor evolution studies. In this example, immunocompromised zebrafish were generated that allow robust engraftment of human cells. Specifically, optically-clear homozygous mutant zebrafish that lack the rag2 (recombination activating gene 2) gene and harbor inactivating point mutations in the interleukin-2 receptor gamma a (il2rga) gene (Il2rga^{Y91fs}) were generated. These fish are denoted in this application as rag2^{null/null}; Il2rga−/− zebrafish (see FIG. 1A). In these animals, ˜3.3 kb of the rag2 gene was deleted through CRISPR/Cas9 genome editing (FIG. 1A). This is a significant improvement as compared to the previously reported rag2^{E450fs} mutant animals that have a truncated protein starting from amino acid E450, and results in only a partial and highly pleiotropic immune cell deficiency. In fact, the rag2^{E450fs} mutant is hypomorphic and retains partial abilities to recombine the TCR and immune-globulin loci. The rag2^{null/null}, il2rga−/− fish were created by incrossing rag2^{null/+}; il2rga+/− animals. Progenies from these crosses were scale genotyped at 3 months of age, with rag2^{null/null}, il2rga−/− animals generated at the expected Mendelian ratios.

Single cell sequencing of kidney marrow from rag2^{null/null}; Il2rga−/− mutant animals, the hematopoietic organ in adult zebrafish, revealed a drastic decrease in adaptive immune T and B cells and natural killer cells (FIG. 1B). Using optimized husbandry and intraperitoneal cell transplantation approaches, the rag2^{null/null}; Il2rga−/− zebrafish robustly engrafted a variety of human blood and cancer cells, including CD8+ T cells, rhabdomyosarcoma, glioma, and lymphoma. These animals also successfully engrafted tumor types that failed to efficiently grow in homozygous prkdc^{D3612fs}, il2rga^{Y91fs} zebrafish (denoted herein as prkdc−/−, il2rga−/− zebrafish), such as ovarian cancer, small cell lung cancer, cholangiocarcinoma and gastroadenocarcinoma (FIG. 1C-FIG. 1F). The rag2^{null/null}; Il2rga−/− animals also engraft human peripheral mononuclear blood cells (FIG. 1G).

In addition, rag2^{null/null}; Il2rga−/− animals were tolerant to irradiation (they withstood 20 gy gamma-irradiation; FIG. 111) and hence can be used in the context of hematopoietic stem cell transplantations. prkdc−/−, il2rga−/− immunocompromised animals did not survive the procedure because of the defective prkdc gene (FIG. 1H). Gamma-irradiation kills marrow-derived blood cells, a pre-requisite for hematopoietic stem cell transplants.

Assessing Olaparib and Temozolomide Resistance in Patient Derived Xenograft Models

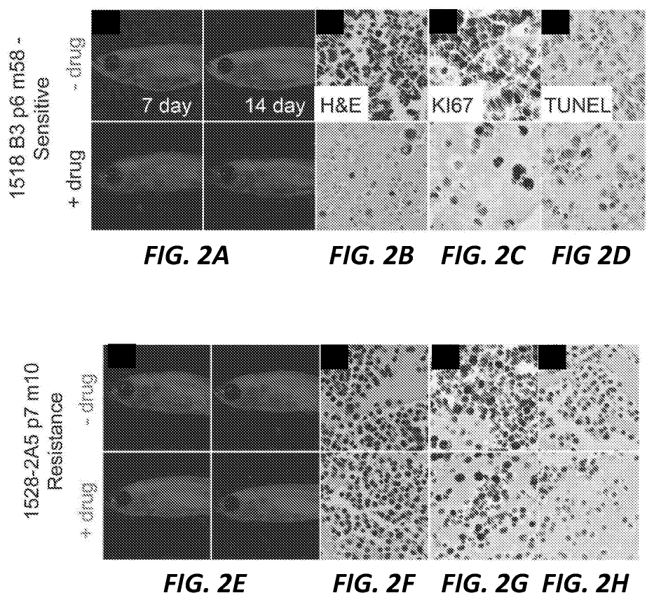

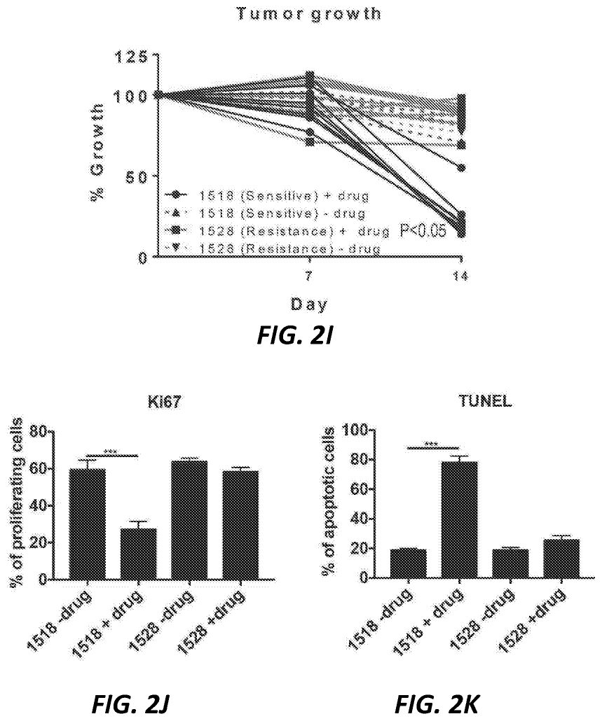

Owning to the superior engraftment capabilities of the rag2^{null/null}; Il2rga−/− animals, small cell lung cancer (SCLC) patient derived xenografts (PDXs) were able to be engrafted into the 3-month-old mutant zebrafish. In mice, experiments involving xenotransplantation of PDXs typically span long time periods, where recipient mice are routinely measured for tumor growth by manual inspection, either visually or by caliper measurement. Leveraging the optical clarity of the mutant zebrafish, approaches to rapidly quantify PDX engraftment and growth were developed. Specifically, by staining these PDXs with long term lineage tracing dyes like CSFE, human cell engraftment for >14 days can easily be visualized. Transplanted cells grown in immunocompromised zebrafish can be directly visualized and quantified by epifluorescence stereomicroscopy imaging and intensity analysis. In this experiment, the drug responsiveness of 2 different PDXs (1518-B3 and 1528-2A5) were tested. 1518-B3 is derived from a SCLC patient that is responsive to the combination of a PARP1 inhibitor, olaparib, and chemotherapy agent temozolomide. In contrast, PDX 1528-2A5 is derived from a SCLC patient that has developed resistance to the combination therapy. Both SCLC PDXs robustly engrafted into rag2^{null/null}; Il2rga−/− zebrafish. Tumors efficiently grew in vehicle control-treated fish, denoted by retention of CSFE and high KI67 and low TUNEL staining of tumors analyzed on section at the end of the experiment. In contrast, when engrafted animals are orally gavaged with clinically relevant doses of olaparib and temozolomide, only animals transplanted with 1518-B3 showed a decreased tumor growth as assessed by reduced CSFE intensity, and which was further validated by increased apoptosis and decreased cell proliferation following histological analysis. Mutant animals receiving PDX 1528-2A5 were unresponsive to treatment, consistent with clinical responses observed in the patients from which the PDXs are derived from (FIG. 2A-FIG. 2K).

Using Rag2^{null/null}; Il2rga−/− Zebrafish as a Tool to Evaluate Immunotherapy Efficiency

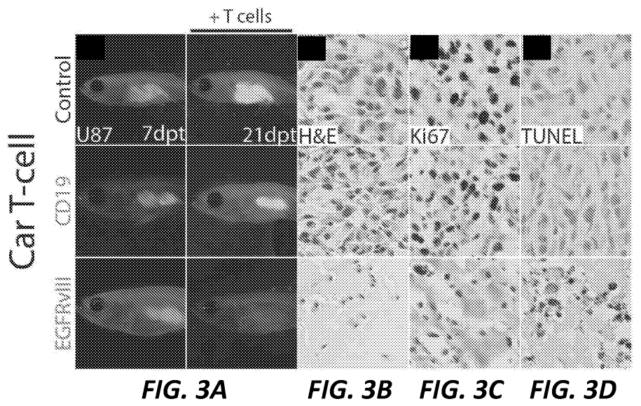

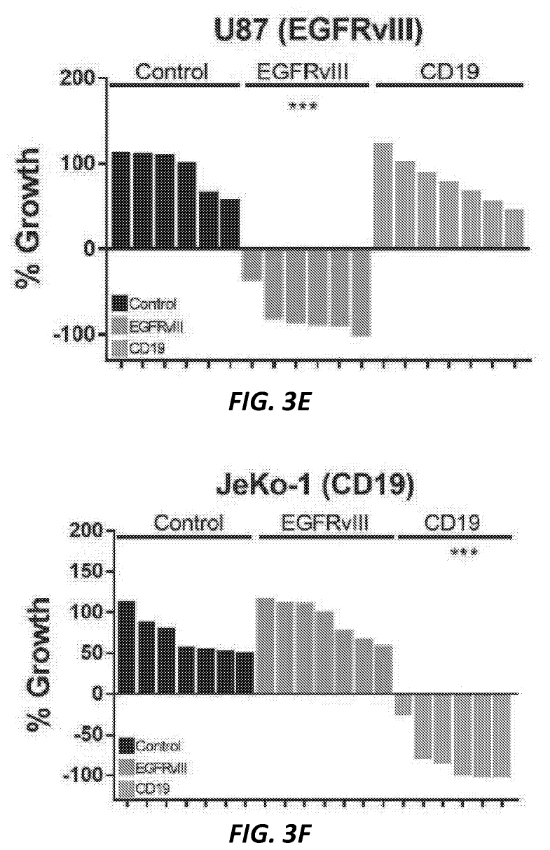

A significant improvement of the rag2^{null/null}; Il2rga−/− zebrafish model is that it robustly engrafts human blood cells (FIG. 1G) and T cells (FIG. 1E). In this example, this model was used to investigate efficiencies of three different types of T-cell mediated immunotherapies: Chimeric Antigen Receptor T cell therapy (CAR-T), Bispecific T cell engager therapy (BiTE), and Antibody peptide epitope circuit therapy (APEC). See FIGS. 3A-3F, 4A-4F, 5A-5F, 6A-6E, 7A-7E, 8A-8E, 9A-9C, and 10A-10J.

Evaluating Car T-Cell Therapy In Vivo and Visualizing Responses at Single Cell Resolution

To investigate if rag2^{null/null}; Il2rga−/− animals can be used to evaluate efficiencies of Car T-cell therapies, an EGFRvIII-expressing glioma cell line (U87) was transplanted into the intraperitoneal cavity of the immunocompromised zebrafish. These U87 cells were also lentiviral transfected with GFP to allow direct visualization of tumor engraftment using epifluorescence stereomicroscopy. Following engraftment of tumor cells for 7 days, these animals are injected intraperitoneally with either untransduced CD8+ T-cells, non-specific CD19 targeting Car T-cells or Car T-cells targeting EGFRvIII antigen. In both types of control (untransduced and non-specific), tumor cells continued to grow as denoted by increased fluorescence intensity which was confirmed on histological analysis showing high proliferation capacity and overall low rates of apoptosis. By contrast, administering target specific EGFRvIII Car T-cells robustly suppressed tumor growth, which was confirmed histologically by both decreasing cell proliferation and increasing apoptosis. In addition, the same study was repeated using CD19 Car T-cells in CD19-expressing Jeko-1 lymphoma cells. As expected, CD19 CAR-T cells robustly killed engrafted Jeko-1 cells while EGFRvIII CAR-T cells did not. These experiments validated the model as a useful tool for investigating potency both in cell killing and specificity of Car T-cell immunotherapy targeting (FIG. 3A-FIG. 3F).

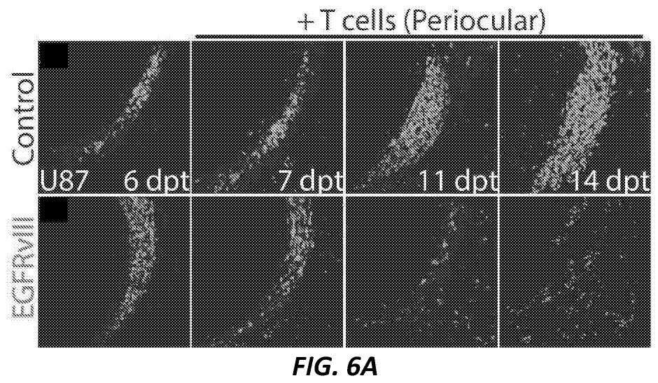

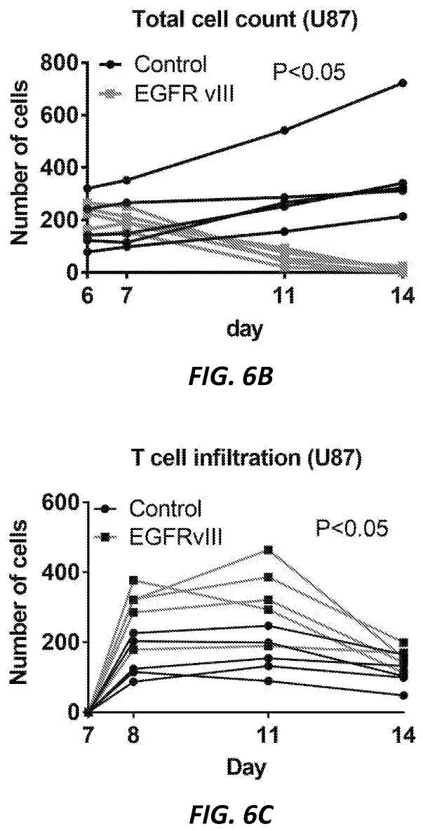

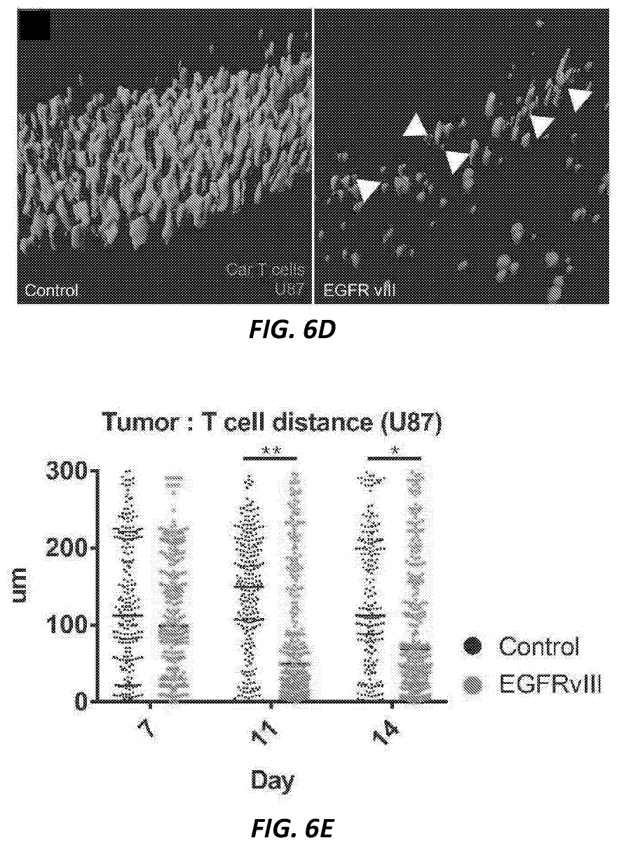

The mutant rag2^{null/null}; Il2rga−/− animals were generated in a pigment-lacking, optically-clear strain of zebrafish (Casper), allowing single cell resolution imaging without the need for invasive intravital imaging procedures often required in mice. Here, GFP-expressing U87 cells were engrafted peri-ocularly in the zebrafish, a superficial site that is amendable to confocal microscopic imaging. Next mCherry+ expressing EGFRvIII Car T cells (FIG. 6A-FIG. 6E) were IP injected. Sequential and active migration of Car T cells from the intraperitoneal cavity into the peri-ocular region was documented, followed by a rapid decrease in the overall numbers of tumor cells. 3D modeling of the z-stack confocal images revealed the relative spatial relationship between Car T cells and tumor cells. In the control group, control T cells were unable to infiltrate the tumor mass and were located along the peripheral edges of engrafted tumor mass. By contrast, Car T-cells that harbored the correct antigen recognition were able to infiltrate the tumor and were almost always positioned in close proximity of tumor cells (FIG. 6D-FIG. 6E).

Evaluating BiTE Therapy and Quantifying In Vivo Cell Killing at Single Cell Resolution

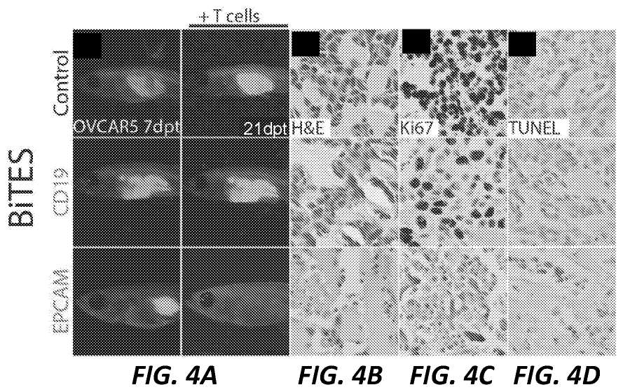

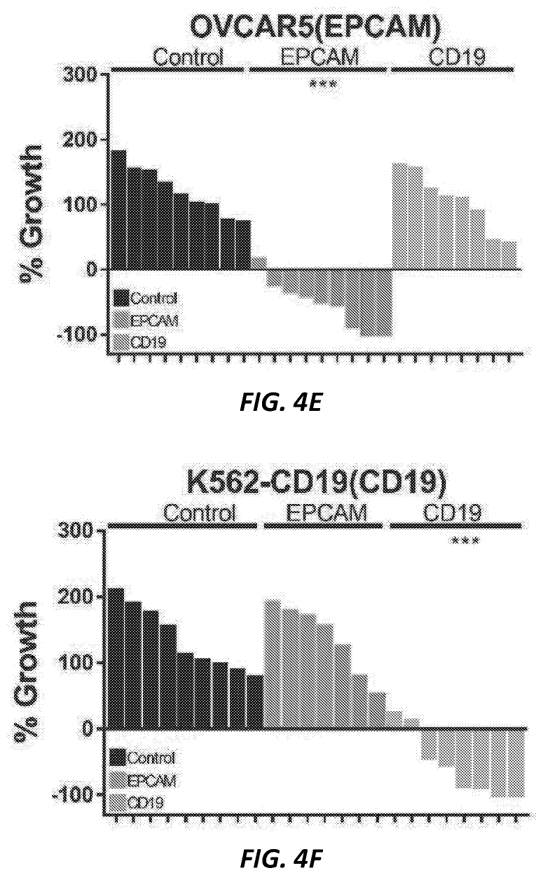

To investigate if the rag2^{null/null}; Il2rga−/− animals can be used in the context of evaluating BiTE therapy efficiency, GFP-expressing OVCAR-5 (EPCAM+) cancer cells were first engrafted into mutant zebrafish. Following 7 days of engraftment, CD8+ T cells were co-administered with a control EPCAM antibody, a non-specific CD19 targeting BiTE (blinatumomab), or an EPCAM targeting BiTE (solitomab). Co-administration of CD8+ T cells with control EPCAM antibody or blinatumomab did not alter overall OVCAR-5 tumor growth. Only with the co-administration of solitomab did OVCAR-5 tumor cells undergo cell killing and suppress tumor growth. In a similar experiment, animals engrafted with CD19-expressing leukemia cell line (K562-CD19) cells were killed off by coadministration of CD8+ T cells and blinatumomab (FIG. 4A-FIG. 4F).

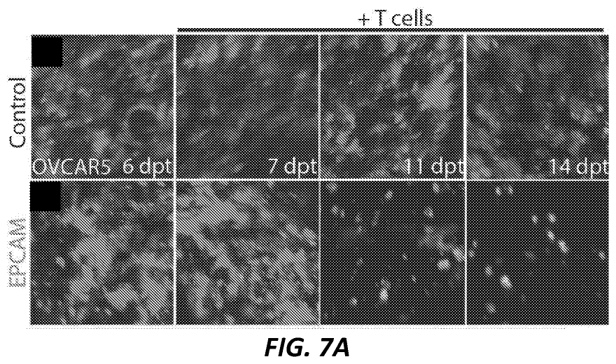

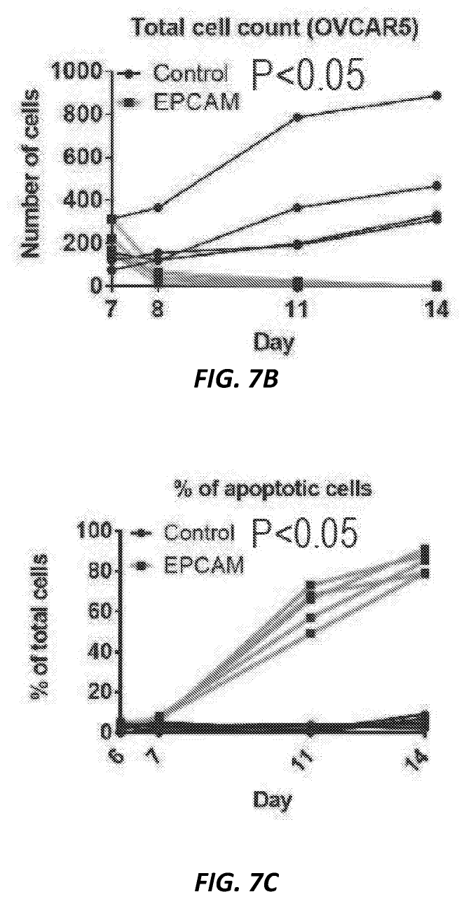

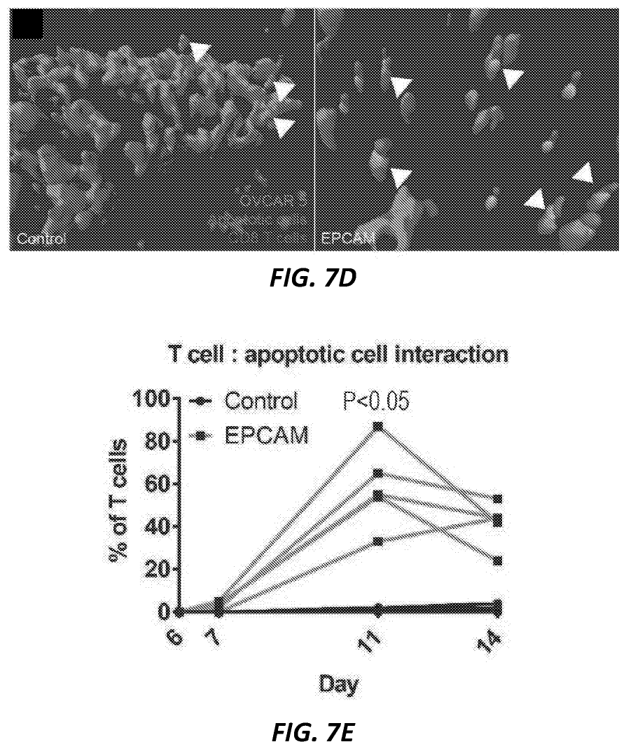

Next, it was investigated whether the rag2^{null/null}; Il2rga−/− zebrafish are able to be used to directly visualize tumor cell killing at single cell resolution following BiTE therapy in vivo. To do so, OVCAR-5 ovarian cancer cells were engineered to express a zipCaspase 3 construct, where live tumor cells constitutively express mCherry, but express zipGFP only when undergoing apoptosis (FIG. 7A-FIG. 7E). By live confocal microscopy imaging of engrafted OVCAR-5 (zipCaspase3) cells administered with solitomab and T cells, a significant induction of cell apoptosis was able to be quantified in vivo and in real time (FIG. 7B, FIG. 7C, FIG. 7D, and FIG. 7E). Further modelling of 3D confocal images revealed that apoptotic OVCAR-5 cells were always in direct contact with a CD8+ T cells, validating a model by which the BiTE antibody redirects T cells to interact and kill tumor cells (FIG. 7D).

Evaluating APEC Therapy Efficiency and Quantifying Target Specificity

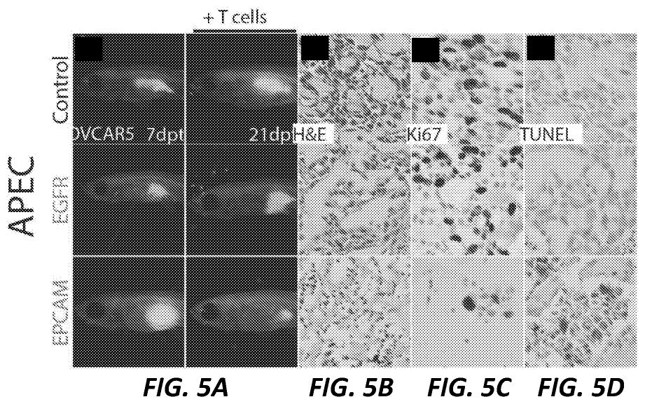

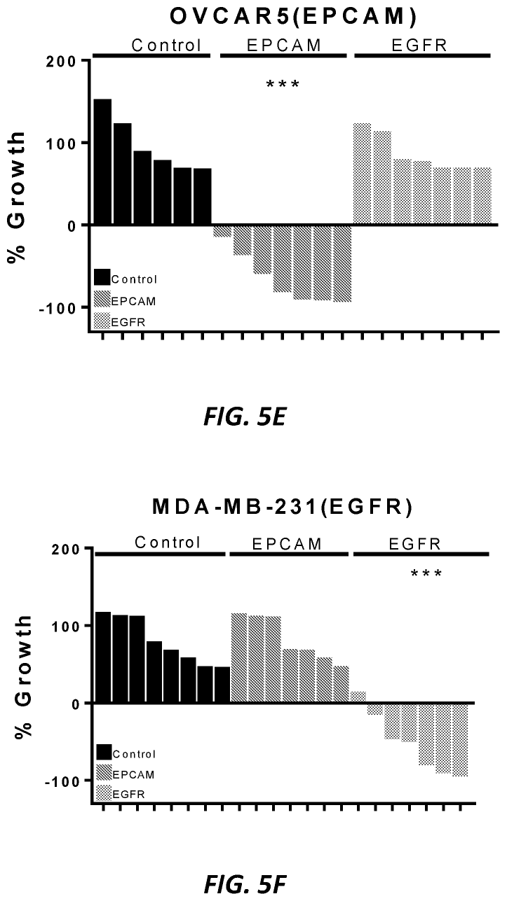

Lastly, the model was applied in the context of a novel preclinical APEC immunotherapy. Basic principles of the APEC therapy are as such: an antibody targeting tumor specific antigen is modified by covalently attaching a peptide chain to the antibody. The attached peptide chain consists of a protease cleavage site that is cut by tumor specific protease, as well as a CMV viral epitope that binds to MHC I receptor on the tumor cell, tricking T cells into attacking tumor cells. Following engraftment of EPCAM+OVCAR5 cells into our animals, CMV primed T cells was co-administered with either control EPCAM antibody, non-specific EGFR APEC or EPCAM specific APECs. As expected, administration of CMV-primed T cells with control EPCAM antibody or non-specific APEC (EGFR) failed to suppress tumor growth, but only target specific EPCAM APECs, together with CMV-primed T cells, potently killed engrafted OVCAR-5 cells (FIG. 5A-FIG. 5F). In addition, a separate experiment further demonstrated that EGFR targeting APECs can robustly kill engrafted breast cancer MDA-MB-231 cells.

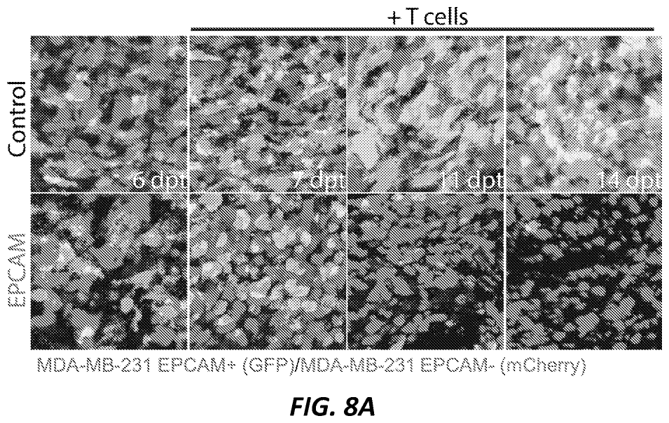

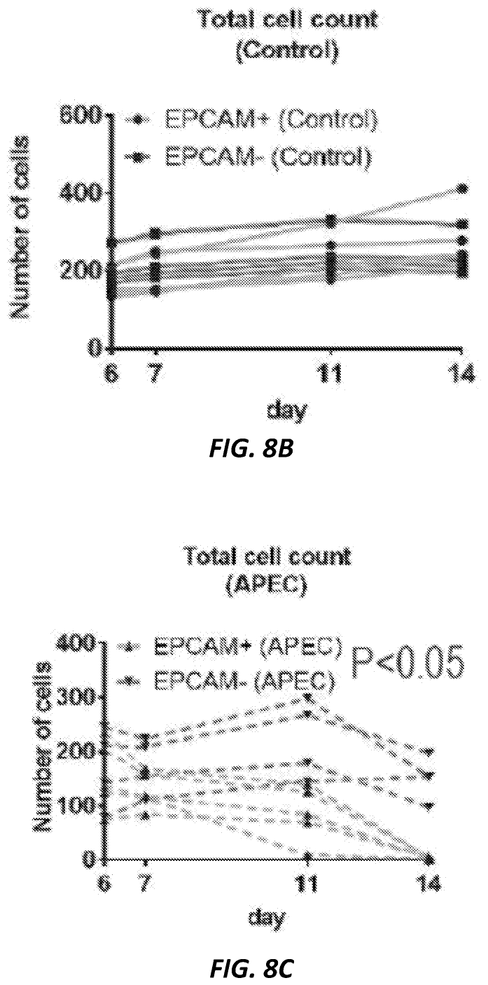

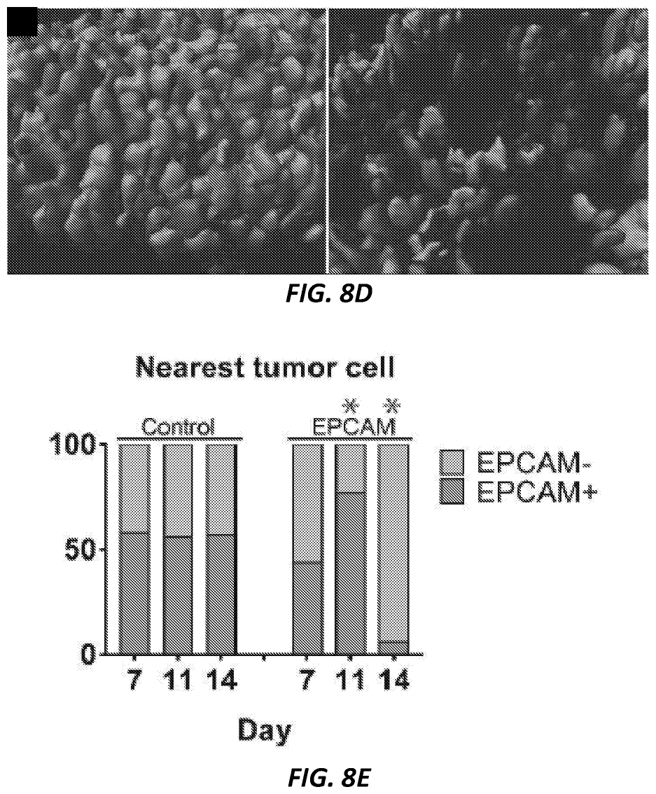

To further test if the rag2^{null/null}; Il2rga−/− animals can be used to visualize the target specificity of APEC immunotherapy, GFP expressing MDA-MB-231 cells that were engineered to express EPCAM were co-engrafted together with wild type mCherry+MDA-MB-231 cells that do not express EPCAM, and injected into the rag2^{null/null}; Il2rga−/− animals (FIG. 8A-FIG. 8E). With the co-administration of EPCAM APEC with T cells, rapid and selective killing of EPCAM+MDA-MB-231 cells was seen, which were nearly completely ablated after 4 days of therapy administration (FIG. 8A-FIG. 8E). 3D modeling of tumor cells showed selective contact of T cells with only GFP-expressing cells but not tumor cells that do not express the antigen (FIG. 8D, FIG. 8E).

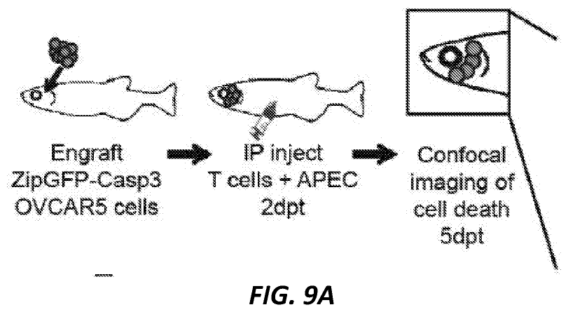

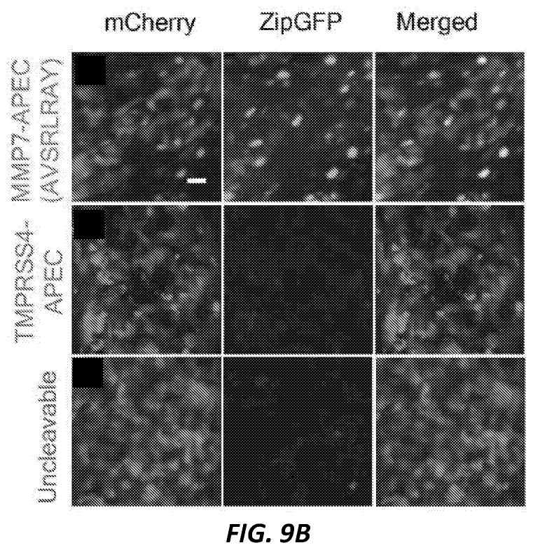

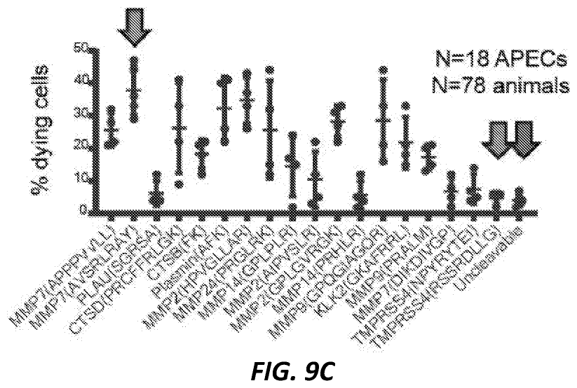

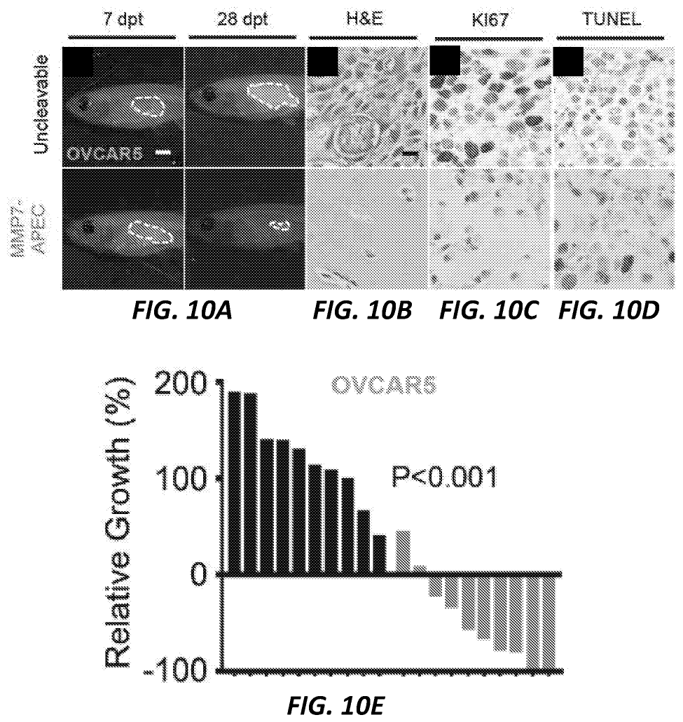

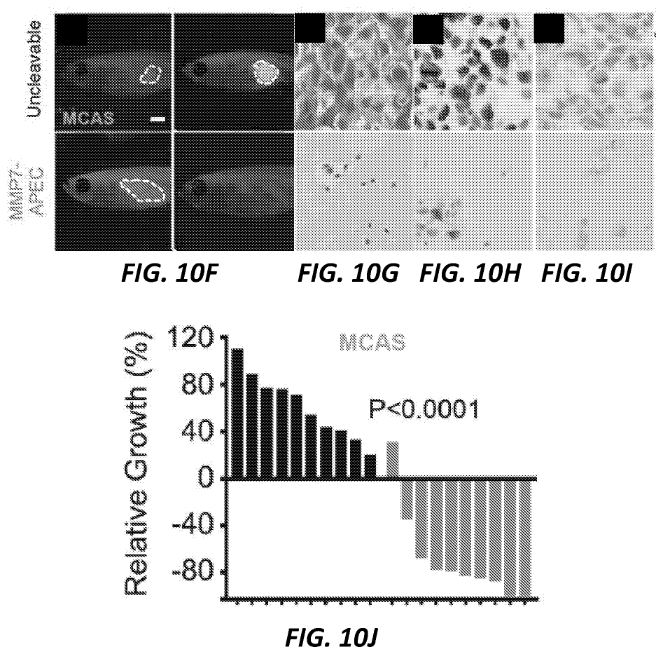

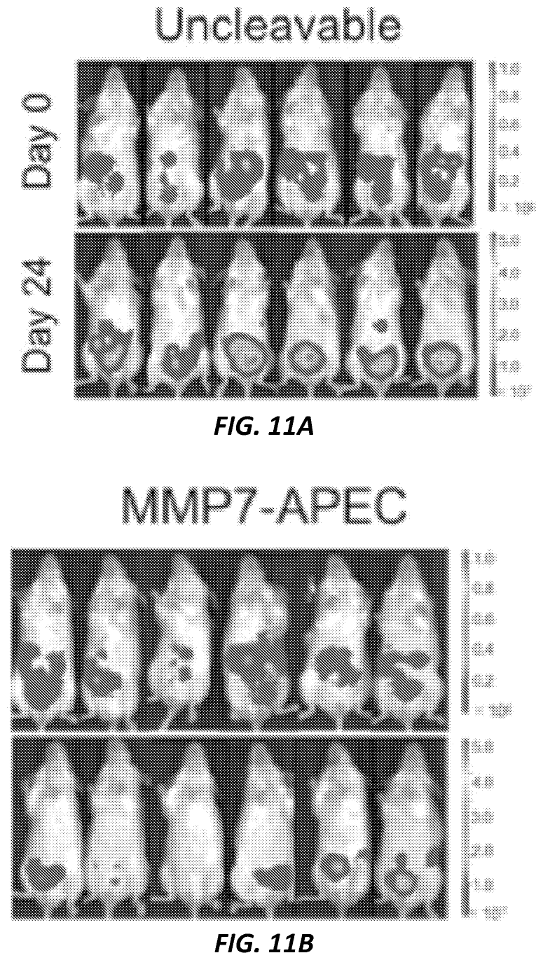

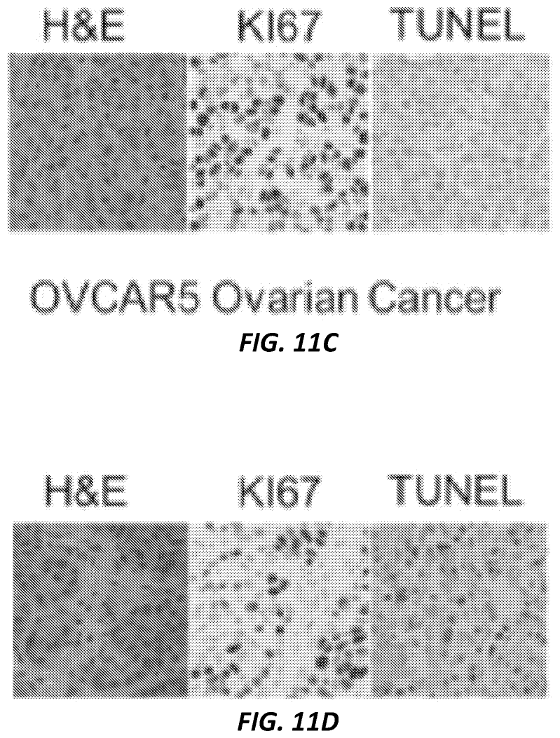

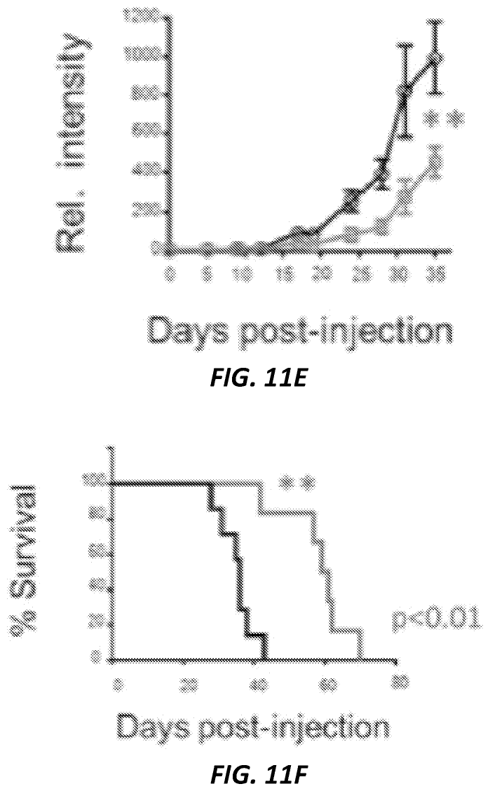

Zebrafish is a model organism that is inherently suitable for high throughput studies, largely due to the low husbandry and maintenance cost. To demonstrate the power of this model in the context of a high throughput drug discovery study, the killing efficiency of 18 different APEC antibodies in OVCAR-5 cells that expresses the zipCaspase3 construct was investigated. In a rapid 5 day assay, using percentage of apoptotic cells as a readout, the OVCAR-5 cells were engrafted peri-ocularly into the animal and the different APECs along with T cells were co-administered. These animals were imaged by confocal microscopy and the number of apoptotic cells quantified. From this study, the best vivo APEC that elicits OVCAR-5 killing (MMP7(AVSRLRAY)-APEC (abbreviated herein as MMP7-APEC, FIG. 9A-FIG. 9C) was identified. A longer-term study evaluating the effect of MMP7-APEC on ovarian cancer cell growth further validated the effectiveness of this APEC in zebrafish (FIG. 10A-FIG. 10J) and mouse xenograft studies (FIG. 11A-FIG. 11F).

Conclusion

The casper-strain rag2^{null/null}; Il2rga−/− zebrafish described in this example can robustly engraft a wide array of human cancers and T cells, can be reared at 37° C., and permit long-term, single-cell visualization of cancer cell engraftment and responses to therapy. Building on the optical clarity of the model, a variety of cell labeling approaches were used to evaluate drug discovery, resistance and immunotherapy responses at single cell resolution in vivo that will extend to a wide array of cancer types and zebrafish immune deficiency models. As compared to previous iterations of immunocompromised zebrafish, the rag2^{null/null}; Il2rga−/− animals are more severely immunodeficient, engraft new cancer types that failed in previous models, are tolerant to irradiation and hence can be used for hematopoietic stem cell transplantation, and can robustly engraft human T cells.

The ability of this model to stably engraft cancers from patient derived xenografts is also particularly exciting. Large-scale pre-clinical testing using a patient's own tumor to aid may be used in the stratifying patients into trials for which their tumor is best predicted to respond. When coupled to the inherent advantage of the zebrafish model for large-scale, high-throughput drug screening, it is be possible to perform combination drug screening using oral-gavage to achieve accurate, clinically-relevant delivery. The rag2^{null/null}; Il2rga−/− zebrafish as has been established herein as a useful tool for evaluating efficiencies of immunotherapy. The model is highly amendable to single cell resolution in vivo imaging and allowed for watching the interactions between T cells with tumor cells, testing killing efficiencies in vivo at real time, and rapidly evaluating target specificity.

### Example 2. Other Immunocompromised Zebrafish Models for Xenotransplantation of Human Cells

In addition to rag2^{null/null}; Il2rga−/− zebrafish, zebrafish mutants have been generated with truncated il2rgb gene (il2rgb^{D33fs}) (FIG. 12B) and nk-lysin gene deletion (nk-lysin^{null/null}) (FIG. 12A). Homozygous il2rgb^{D33fs} or nk-lysin^{null/null} zebrafish were viable as homozygous adult animals, but as single homozygous mutant animals did not efficiently engraft human cells. The aim of generating these animals was to create compound, quadruple mutant zebrafish of rag2^{null/null}; nk-lysin^{null/null}; Il2rga−/−; Il2rgb−/−, or prkdc−/−; nk-lysin^{null/null}; Il2rga−/−; Il2rgb−/−, which have further reduced T, B, and NK cell function and provide superior engraftment capabilities over currently described models.

Specifically, using CRISPR-Cas9 mediated gene deletion, ˜43 kb of the nk-lysin1-4 gene locus was deleted. NK-lysins are granzyme proteins that are likely responsible for cytotoxic cell killing. Single cell sequencing on both prkdc−/−; Il2rga−/−, as well as rag2^{null/null}; Il2rga−/− mutant zebrafish kidney marrow showed retention of a small population of NK-lysin positive cells, which suggests targeting these cells may enhance overall long-term engraftment rates. Zebrafish have 2 orthologue copies of the human il2rg gene, il2rga and il2rgb. Mutating il2rgb eliminates possibilities of compensatory expression of il2rgb gene with the mutation of il2rga.

Viable, quadruple mutant prkdc−/−; nk-lysin^{null/null}; Il2rga−/−; Il2rgb−/− were created and used to efficiently engraft human cancer (see Table below).

| Mutants | Viability | Survival | Engraftment |
|---|---|---|---|
| NK-lysin^{null/null} | Viable to | Similar survival | Single |
| | adulthood | and life span as | homozygous |
| | under normal | wild type | mutant fish do not |
| | husbandry | zebrafish, >1 year | engraft human |
| | conditions | with normal | cancer cells when |
| | | husbandry | grown at 37° C. |
| | | condition | |
| il2rgb^{D33fs}−/− | Viable to | Similar survival | Single |
| | adulthood | and life span as | homozygous |
| | under normal | wild type | mutant fish do not |
| | husbandry | zebrafish, >1 year | engraft human |
| | conditions | with normal | cancer cells when |
| | | husbandry | grown at 37° C. |
| | | condition | |
| prkdc^{D3612fs}−/−; | Viable to | >6 months | Compound mutant |
| NK-lysin^{null/null}; | adulthood | survival when | animals efficiently |
| il2rga^{Y91fs}−/−; | following | grown in | engraft Human RD |
| il2rgb^{D33fs}−/− | rearing | antibiotics- | cells (4/4 animals) |
| | in antibiotics- | supplemented | in excess of 28 |
| | supplemented | fish water | days when grown |
| | fish water | | at 37° C. |

Crosses needed to generate rag2^{null/null}; nk-lysin^{null/null}; Il2rga−/−; Il2rgb−/− animals are performed.

Methods

Zebrafish

rag2^{null/null} mutants were created in casper-strain zebrafish using CRISPR-Cas9 mediated gene deletion. Guide RNA sequence are 5′ gsRNA-AGAACCGTATCAAGCGCGGG (SEQ ID NO:11) and 3′ gsRNA-GGCCCTTGACTACATATGGTG (SEQ ID NO: 12).

NK-lysin^{null/null} mutants were created in casper-strain zebrafish using CRISPR-Cas9 mediated gene deletion. Guide RNA sequence are 5′ gsRNA-GGTTCTGGCATTATCTGTAG (SEQ ID NO:13) and 3′ gsRNA-AGCAGCAAGTAGTCGAGCAG (SEQ ID NO: 14).

il2rga^{Y91fs} mutants were created in casper-strain zebrafish using TALEN-mediated mutagenesis. TALEN targeting sequences were

(SEQ ID NO: 15)
5′-TACATTGAAAACAAGCCT-3′
and
(SEQ ID NO: 16)
5′-AGTTTGGTGACGGAACAGGA-3′.

il2rgb^{D33fs} mutants were created in casper-strain zebrafish using TALEN-mediated mutagenesis. TALEN targeting sequences were

(SEQ ID NO: 17)
5′-TGYGCAGTGTAAAATCAT-3′
and
(SEQ ID NO: 18)
5′-AGTGCATCTGGCAACGGA-3′.

Prkdc^{D631fs} mutants were created in casper-strain zebrafish using TALEN-mediated mutagenesis. TALEN targeting sequences were

(SEQ ID NO: 19)
5′-GACTGAATTGCTT-3′
and
(SEQ ID NO: 20)
5′-AAGATTTGGGTCTTACAGA-3′.

Specifically, RNA was prepared for each pair of gRNA (rag2 and NK-lysin) with CAS9 mRNA or each pair of TALEN arm (il2rga and il2rgb) and microinjected into 1 cell stage embryos from casper zebrafish. F₀-injected animals were raised to adulthood, and single male-by-female matings performed. 12 resultant progeny from each cross were arrayed into 96-well plates and genomic DNA was extracted. PCR amplification of the target sites was performed, and amplicons were sent for amplicon sequencing. From this analysis, one mutant line for each rag2, NK-lysin, il2rga or il2rgb gene was identified. F₁ progeny were subsequently raised to adulthood, and heterozygous fish were identified by genotyping.

Identified heterozygous rag2^{null/+} and il2rga^{Y91fs}+/− mutants were crossed to generate compounded mutant animals rag2^{null/+}; il2rga^{Y91fs}+/− animals, which were incrossed to generate double homozygous rag2^{null/null}; il2rga^{Y91fs}−/− animals.

Identified heterozygous NK-lysin^{null/+}, il2rga^{Y91fs}+/− and il2rgb^{D33fs}+/− mutant animals were sequentially crossed with prkdc^{D3612fs}−/− animals, generating quadruple prkdc^{D3612fs}+/−; NK-lysin^{null/+}; il2rga^{Y91fs}+/−; il2rgb^{D33fs}+/−; animals, which were incrossed to generate quadruple prkdc^{D3612fs}−/−; NK-lysin^{null/null}; il2rga^{Y91fs}−/−; il2rgb^{D33fs}−/−animals.

Human Cancer Cell and T Cell Transplantation into Zebrafish

Human cancer cells were grown to 90% confluence in a T75 cell culture flask, trypsinized, counted and resuspended at 2.5×10⁷ cells/ml in Matrigel. Clondronate liposomes were added to the injection mix to inhibit early macrophage ingestion of engrafted cells over the first 7 days. 20 μl of volume was injected into the peritoneal cavity of recipient fish using 30½ gauge needle (5×10⁵ cells/fish). For ocular muscle injections, 2 μl of cell suspension was injected (5×10⁴ cells/fish). Human CD8+ T cells, CMV-primed T cells and Car T cells were grown in 24 well plate at a density of 1×10⁶ cells/ml, harvested, counted and resuspended at 5×10⁷ cells/ml in T cell media. 5 μl of volume was injected into the peritoneal cavity of recipient fish using 30½ gauge needle (2.5×10⁵ cells/fish). Recipient zebrafish were then raised at 37° C. in sterilized fish water containing the aforementioned antibiotics. Cancer cell engraftment was assessed using epifluorescence whole animal microscopy (Olympus MVX10), with average GFP intensity measured using ImageJ. Growth was measured in relation to GFP intensity observed immediately after IP injection or by individual count of GFP+ cells over time. CD8+ T cell engraftment was visualized by CD3 antibody staining of peripheral blood extracted from recipient animals at various time points. Recipient fishes were sacrificed when moribund or at ≥28 dpt, fixed in 4% paraformaldehyde, and sectioned for histological examination. Similar approaches were used for engraftment of cancer cells labeled with other fluorescent-reporters and PDX tumors.

Assessing Immunotherapy Responses in Zebrafish Xenografts

All transplanted human CD8+ T cells, CMV-primed T cells and Car T cells are stained with 1 uM lineage tracing dye, CSFE, for 15 min, prior to transplantation. Engrafted fish were selected at 7 days post transplantation, and transplanted with T cells into intra-peritoneal cavity. In recipient animals engrafted with tumor cells in the intra-peritoneal cavity, animals are imaged by stereo epifluorescence microscopy on day 0, 7, 14 and 21 day post transplantation. Engrafted zebrafish with peri-ocular engraftment of tumor cells are imaged by confocal microscopy on day 6, 7, 11 and 14 day post transplantation. For assessing Car T cell therapy responses, Control CD8+ T cells, CD19 Car T cells or EGFRvIII Car T cells were IP injected into the animals. For analysis of BiTE therapy responses, CD8+ T cells, with either control EPCAM, control CD19 antibody (0.25 mg/kg), blinatumomab (0.25 mg/kg) or solitomab (0.05 mg/kg) were IP injected into animals. For analysis of APEC therapy responses, CMV-primed T cells, with either control EPCAM, control EGFR antibody (2.5 mg/kg), EGFR APEC (2.5 mg/kg) and EPCAM APEC (2.5 mg/kg) were IP injected into the animals. Quantification of therapy response including total cell count, 3d distance mapping for animals engrafted peri-ocularly with tumor cells are carried out using Imaris or ImageJ.

Confocal Imaging and Quantitation.

Imaging of peri-ocular transplanted cells was performed using an inverted LSM 710 confocal microscope. Engrafted zebrafish were anesthetized using 168 mg/ml tricaine and placed into a 12-mm glass-bottom dish for imaging. Serial z-stack imaging was carried out using a 10× objective (NA 0.45). In vivo direct visualization of Car T-cell interaction with tumor cells, cell apoptosis with zipGFP caspase 3 reporter, and visualization of therapy target specificity was carried out by serial z-stack imaging using 405 nm (emission 360-480 nm), 488 nm (emission=493-586 nm) and 546 nm laser (emission=575-703 nm). Animals were imaged at 6 dpt prior to administration of immunotherapy. Subsequent imaging was completed at 7, 11 and 14 day post transplantation. 3-dimensional modelling analysis of captured z-stack confocal images was performed using Imaris 11.0. Quantification of distance between tumor cells and Car T-cells was analysed using distance between surface and spot plugin. Images were manually annotated from Imaris files (n=100 cells per timepoint analyzed from n≥3 animals) and quantified using the Fisher's Exact Test or T-test.

Assessing Small Cell Lung Cancer PDX Responses in Zebrafish Xenografts

Small cell lung cancer PDXs were harvested from xenografts passaged subcutaneously in NSG mice, dissociated into single cell suspension, stained with lineage tracing dye CSFE, and transplanted intra-peritoneally. Engrafted fish were selected at 7 days post injection and orally gavaged with 50 mg/kg of Olaparib and 25 mg/kg of temozlomide (vehicle was 1% DMSO in 1×PBS). Control vehicle treated fish were orally gavaged with 1% DMSO in 1×PBS. Gavage was performed using a Hamiliton 22 G needle and 22 G soft-tip catheter tubing. Drugs were orally administered at 7 dpt, and recipient fishes imaged before drug administration and at the end of experiment (14 dpt). At end of experiment, animals were fixed in 4% paraformaldehyde, sectioned, and examined histologically.

### REFERENCES

1. Barberi, T., et al., Derivation of engraftable skeletal myoblasts from human embryonic stem cells. Nat Med, 2007. 13(5): p. 642-8.
2. Cooper, R. N., et al., A new immunodeficient mouse model for human myoblast transplantation. Hum Gene Ther, 2001. 12(7): p. 823-31.
3. Ehrhardt, J., et al., Human muscle precursor cells give rise to functional satellite cells in vivo. Neuromuscul Disord, 2007. 17(8): p. 631-8.
4. Huard, J., et al., High efficiency of muscle regeneration after human myoblast clone transplantation in SCID mice. J Clin Invest, 1994. 93(2): p. 586-99.
5. Negroni, E., et al., In vivo myogenic potential of human CD133+ muscle-derived stem cells: a quantitative study. Mol Ther, 2009. 17(10): p. 1771-8.
6. Dick, J. E., et al., In vivo dynamics of human stem cell repopulation in NOD/SCID mice. Ann N Y Acad Sci, 2001. 938: p. 184-90.
7. Guenechea, G., et al., Distinct classes of human stem cells that differ in proliferative and self-renewal potential. Nat Immunol, 2001. 2(1): p. 75-82.
8. Majeti, R., C. Y. Park, and I. L. Weissman, Identification of a hierarchy of multipotent hematopoietic progenitors in human cord blood. Cell Stem Cell, 2007. 1(6): p. 635-45.
9. McDermott, S. P., et al., Comparison of human cord blood engraftment between immunocompromised mouse strains. Blood, 2010. 116(2): p. 193-200.
10. Notta, F., et al., Isolation of single human hematopoietic stem cells capable of long-term multilineage engraftment. Science, 2011. 333(6039): p. 218-21.
11. Park, C. Y., R. Majeti, and I. L. Weissman, In vivo evaluation of human hematopoiesis through xenotransplantation of purified hematopoietic stem cells from umbilical cord blood. Nat Protoc, 2008. 3(12): p. 1932-40.
12. Tanaka, S., et al., Development of mature and functional human myeloid subsets in hematopoietic stem cell-engrafted NOD/SCID/IL2rgammaKO mice. J Immunol, 2012. 188(12): p. 6145-55.
13. Racki, W. J., et al., NOD-scid IL2rgamma(null) mouse model of human skin transplantation and allograft rejection. Transplantation, 2010. 89(5): p. 527-36.
14. van Laake, L. W., et al., Human embryonic stem cell-derived cardiomyocytes survive and mature in the mouse heart and transiently improve function after myocardial infarction. Stem Cell Res, 2007. 1(1): p. 9-24.
15. Kroon, E., et al., Pancreatic endoderm derived from human embryonic stem cells generates glucose-responsive insulin-secreting cells in vivo. Nat Biotechnol, 2008. 26(4): p. 443-52.
16. Cheng, X., et al., Self-renewing endodermal progenitor lines generated from human pluripotent stem cells. Cell Stem Cell, 2012. 10(4): p. 371-84.
17. Yui, S., et al., Functional engraftment of colon epithelium expanded in vitro from a single adult Lgr5(+) stem cell. Nat Med, 2012. 18(4): p. 618-23.
18. Nehls, M., et al., New member of the winged-helix protein family disrupted in mouse and rat nude mutations. Nature, 1994. 372(6501): p. 103-7.
19. Bosma, G. C., R. P. Custer, and M. J. Bosma, A severe combined immunodeficiency mutation in the mouse. Nature, 1983. 301(5900): p. 527-30.
20. Blunt, T., et al., Defective DNA-dependent protein kinase activity is linked to V(D)J recombination and DNA repair defects associated with the murine scid mutation. Cell, 1995. 80(5): p. 813-23.
21. Shultz, L. D., et al., Multiple defects in innate and adaptive immunologic function in NOD/LtSz-scid mice. J Immunol, 1995. 154(1): p. 180-91.
22. Mombaerts, P., et al., RAG-1-deficient mice have no mature B and T lymphocytes. Cell, 1992. 68(5): p. 869-77.
23. Shultz, L. D., et al., NOD/LtSz-Rag1null mice: an immunodeficient and radioresistant model for engraftment of human hematolymphoid cells, HIV infection, and adoptive transfer of NOD mouse diabetogenic T cells. J Immunol, 2000. 164(5): p. 2496-507.
24. Pearson, T., et al., Non-obese diabetic-recombination activating gene-1 (NOD-Rag1 null) interleukin (IL)-2 receptor common gamma chain (IL2r gamma null) null mice: a radioresistant model for human lymphohaematopoietic engraftment. Clin Exp Immunol, 2008. 154(2): p. 270-84.
25. Ishikawa, F., et al., Development of functional human blood and immune systems in NOD/SCID/IL2 receptor {gamma} chain(null) mice. Blood, 2005. 106(5): p. 1565-73.
26. Shultz, L. D., et al., Human lymphoid and myeloid cell development in NOD/LtSz-scid IL2R gamma null mice engrafted with mobilized human hemopoietic stem cells. J Immunol, 2005. 174(10): p. 6477-89.
27. Shultz, L. D., et al., NOD/LtSz-Rag1nullPfpnull mice: a new model system with increased levels of human peripheral leukocyte and hematopoietic stem-cell engraftment. Transplantation, 2003. 76(7): p. 1036-42.
28. Christianson, S. W., et al., Enhanced human CD4+ T cell engraftment in beta2-microglobulin-deficient NOD-scid mice. J Immunol, 1997. 158(8): p. 3578-86.
29. Nosaka, T., et al., Defective lymphoid development in mice lacking Jak3. Science, 1995. 270(5237): p. 800-2.
30. Thomis, D. C., et al., Defects in B lymphocyte maturation and T lymphocyte activation in mice lacking Jak3. Science, 1995. 270(5237): p. 794-7.
31. Boehm, T., C. C. Bleul, and M. Schorpp, Genetic dissection of thymus development in mouse and zebrafish. Immunol Rev, 2003. 195: p. 15-27.
32. Iwanami, N., et al., Genetic evidence for an evolutionarily conserved role of IL-7 signaling in T cell development of zebrafish. J Immunol, 2011. 186(12): p. 7060-6.
33. Wienholds, E., et al., Target-selected inactivation of the zebrafish rag1 gene. Science, 2002. 297(5578): p. 99-102.
34. White, R. M., et al., Transparent adult zebrafish as a tool for in vivo transplantation analysis. Cell Stem Cell, 2008. 2(2): p. 183-9.
35. Blackburn, J. S., S. Liu, and D. M. Langenau, Quantifying the Frequency of Tumor-propagating Cells Using Limiting Dilution Cell Transplantation in Syngeneic Zebrafish. J Vis Exp, 2011(53).
36. Blackburn, J. S., et al., Notch signaling expands a pre-malignant pool of T-cell acute lymphoblastic leukemia clones without affecting leukemia-propagating cell frequency. Leukemia, 2012.
37. Ignatius, M. S., et al., In Vivo imaging of tumor-propagating cells, regional tumor heterogeneity, and dynamic cell movements in embryonal rhabdomyosarcoma. Cancer Cell, 2012. 21(5): p. 680-93.
38. Smith, A. C., et al., High-throughput cell transplantation establishes that tumor-initiating cells are abundant in zebrafish T-cell acute lymphoblastic leukemia. Blood, 2010.
39. Traver, D., et al., Effects of lethal irradiation in zebrafish and rescue by hematopoietic cell transplantation. Blood, 2004. 104(5): p. 1298-305.
40. Hansen, J. D. and A. G. Zapata, Lymphocyte development in fish and amphibians. Immunological Reviews, 1998. 166: p. 199-220.
41. Willett, C. E., et al., Early hematopoiesis and developing lymphoid organs in the zebrafish. Developmental Dynamics, 1999. 214(4): p. 323-36.
42. Willett, C. E., et al., Expression of zebrafish rag genes during early development identifies the thymus. Developmental Biology, 1997. 182(2): p. 331-41.
43. Langenau, D. M., et al., In vivo tracking of T cell development, ablation, and engraftment in transgenic zebrafish. Proceedings of the National Academy of Sciences of the United States of America, 2004. 101(19): p. 7369-74.
44. Trede, N. S., et al., The use of zebrafish to understand immunity.

Immunity, 2004. 20(4): p. 367-79.
45. Haldi, M., et al., Human melanoma cells transplanted into zebrafish proliferate, migrate, produce melanin, form masses and stimulate angiogenesis in zebrafish. Angiogenesis, 2006. 9(3): p. 139-51.
46. Lee, L. M., et al., The fate of human malignant melanoma cells transplanted into zebrafish embryos: assessment of migration and cell division in the absence of tumor formation. Dev Dyn, 2005. 233(4): p. 1560-70.
47. Ghotra, V. P., et al., Automated whole animal bio-imaging assay for human cancer dissemination. PLoS One, 2012. 7(2): p. e31281.
48. Ali, S., et al., Zebrafish embryos and larvae: a new generation of disease models and drug screens. Birth Defects Res C Embryo Today, 2011. 93(2): p. 115-33.
49. Konantz, M., et al., Zebrafish xenografts as a tool for in vivo studies on human cancer. Ann N Y Acad Sci, 2012. 1266(1): p. 124-137.
50. Corkery, D. P., G. Dellaire, and J. N. Berman, Leukaemia xenotransplantation in zebrafish—chemotherapy response assay in vivo. Br J Haematol, 2011. 153(6): p. 786-9.
51. Traver, D., et al., Transplantation and in vivo imaging of multilineage engraftment in zebrafish bloodless mutants. Nature Immunology, 2003. 4(12): p. 1238-1246.
52. Hess, I., et al., Zebrafish model for allogeneic hematopoietic cell transplantation not requiring preconditioning. Proc Natl Acad Sci USA, 2013. 110(11): p. 4327-32.
53. Stoletov, K., et al., High-resolution imaging of the dynamic tumor cell vascular interface in transparent zebrafish. Proc Natl Acad Sci USA, 2007. 104(44): p. 17406-11.
54. Doyon, Y., et al., Heritable targeted gene disruption in zebrafish using designed zinc-finger nucleases. Nat Biotechnol, 2008. 26(6): p. 702-8.
55. Foley, J. E., et al., Targeted mutagenesis in zebrafish using customized zinc-finger nucleases. Nat Protoc, 2009. 4(12): p. 1855-67.
56. Foley, J. E., et al., Rapid mutation of endogenous zebrafish genes using zinc finger nucleases made by Oligomerized Pool ENgineering (OPEN). PLoS One, 2009. 4(2): p. e4348.
57. Meng, X., et al., Targeted gene inactivation in zebrafish using engineered zinc-finger nucleases. Nat Biotechnol, 2008. 26(6): p. 695-701.
58. Sander, J. D., et al., Selection-free zinc-finger-nuclease engineering by context-dependent assembly (CoDA). Nat Methods, 2011. 8(1): p. 67-9.
59. Sander, J. D., et al., Engineering zinc finger nucleases for targeted mutagenesis of zebrafish. Methods Cell Biol, 2011. 104: p. 51-8.
60. Zhu, C., et al., Evaluation and application of modularly assembled zinc-finger nucleases in zebrafish. Development, 2011. 138(20): p. 4555-64.
61. Cade, L., et al., Highly efficient generation of heritable zebrafish gene mutations using homo- and heterodimeric TALENs. Nucleic Acids Res, 2012.
62. Dahlem, T. J., et al., Simple Methods for Generating and Detecting Locus-Specific Mutations Induced with TALENs in the Zebrafish Genome. PLoS Genet, 2012. 8(8): p. e1002861.
63. Huang, P., et al., Heritable gene targeting in zebrafish using customized TALENs. Nat Biotechnol, 2011. 29(8): p. 699-700.
64. Moore, F. E., et al., Improved Somatic Mutagenesis in Zebrafish Using Transcription Activator-Like Effector Nucleases (TALENs). PLoS One, 2012. 7(5): p. e37877.
65. Sander, J. D., et al., Targeted gene disruption in somatic zebrafish cells using engineered TALENs. Nat Biotechnol, 2011. 29(8): p. 697-8.
66. Cifuentes, D., et al., A novel miRNA processing pathway independent of Dicer requires Argonaute2 catalytic activity. Science, 2010. 328(5986): p. 1694-8.
67. Siekmann, A. F., et al., Chemokine signaling guides regional patterning of the first embryonic artery. Genes Dev, 2009. 23(19): p. 2272-7.
68. Blackburn, J. S., et al., High-throughput imaging of adult fluorescent zebrafish with an LED fluorescence macroscope. Nat Protoc, 2011. 6(2): p. 229-41.
69. Macchi, P., et al., Mutations of Jak-3 gene in patients with autosomal severe combined immune deficiency (SCID). Nature, 1995. 377(6544): p. 65-8.
70. Russell, S. M., et al., Mutation of Jak3 in a patient with SCID: essential role of Jak3 in lymphoid development. Science, 1995. 270(5237): p. 797-800.
71. Frank, J., et al., Exposing the human nude phenotype. Nature, 1999. 398(6727): p. 473-4.
72. Noguchi, M., et al., Interleukin-2 receptor gamma chain mutation results in X-linked severe combined immunodeficiency in humans. Cell, 1993. 73(1): p. 147-57.
73. Perryman, L. E., Molecular pathology of severe combined immunodeficiency in mice, horses, and dogs. Vet Pathol, 2004. 41(2): p. 95-100.
74. Ding, Q., et al., DNA-PKcs mutations in dogs and horses: allele frequency and association with neoplasia. Gene, 2002. 283(1-2): p. 263-9.
75. Pham, L. N., et al., Methods for generating and colonizing gnotobiotic zebrafish. Nat Protoc, 2008. 3(12): p. 1862-75.
76. Rawls, J. F., B. S. Samuel, and J. I. Gordon, Gnotobiotic zebrafish reveal evolutionarily conserved responses to the gut microbiota. Proc Natl Acad Sci USA, 2004. 101(13): p. 4596-601.
77. Rawls, J. F., et al., Reciprocal gut microbiota transplants from zebrafish and mice to germ-free recipients reveal host habitat selection. Cell, 2006. 127(2): p. 423-33.
78. Langenau, D. M., et al., Cre/lox-regulated transgenic zebrafish model with conditional myc-induced T cell acute lymphoblastic leukemia. Proc Natl Acad Sci USA, 2005. 102(17): p. 6068-73.
79. Langenau, D. M., et al., Myc-induced T cell leukemia in transgenic zebrafish. Science, 2003. 299(5608): p. 887-90.
80. Langenau, D. M., et al., Co-injection strategies to modify radiation sensitivity and tumor initiation in transgenic Zebrafish. Oncogene, 2008. 27(30): p. 4242-8.
81. Alexander, M. S., et al., Isolation and transcriptome analysis of adult zebrafish cells enriched for skeletal muscle progenitors. Muscle Nerve, 2011. 43(5): p. 741-50.
82. Higashijima, S., et al., High-frequency generation of transgenic zebrafish which reliably express GFP in whole muscles or the whole body by using promoters of zebrafish origin. Dev Biol, 1997. 192(2): p. 289-99.
83. Mosimann, C., et al., Ubiquitous transgene expression and Cre-based recombination driven by the ubiquitin promoter in zebrafish. Development, 2011. 138(1): p. 169-77.
84. Langenau, D. M., et al., Effects of RAS on the genesis of embryonal rhabdomyosarcoma. Genes Dev, 2007. 21(11): p. 1382-95.
85. Leong, I. U. S. et al., Targeted Mutagenesis of Zebrafish: Use of Zinc Finger Nucleases. Birth Defects Research (Part C) 93:249-255 (2011).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

While the invention has been described with reference to preferred embodiments, those skilled in the Art will appreciate that certain substitutions, alterations and/or omissions may be made to the embodiments without departing from the spirit of the invention. Accordingly, the foregoing Description is meant to be exemplary only, and should not limit the scope of the invention.

Any Reference, Patent or Patent Application Publication disclosed in the present application is incorporated by reference herein in its entirety.

What is claimed is:

1. A genetically-modified fish whose genome is homozygous for a first engineered or induced genetic alteration in recombination-activating gene 2 (rag2) and for a second engineered or induced genetic alteration in interleukin 2-receptor gamma a (il2rga);
wherein the first genetic alteration results in an inactivation of both alleles of rag2;
wherein the second genetic alteration results in an inactivation of both alleles of il2rga; and
wherein the genetic alteration in rag2 is amorphic.

2. The genetically-modified fish of claim 1, which has a genotype rag2^{null/null}; il2rga^{Y91fs}−/−.

3. A genetically-modified fish whose genome is homozygous for an engineered or induced genetic alteration in interleukin 2-receptor gamma b (il2rgb);
wherein the genetic alteration results in an inactivation of both alleles of il2rgb.

4. The genetically-modified fish of claim 3, which has a genotype il2rgb^{D33fs}−/−.

5. A genetically-modified fish whose genome is homozygous for an engineered or induced genetic alteration in NK-lysin genes nkla, nklb, nklc, and nkld;
wherein the genetic alteration results in an inactivation of both alleles of the NK-lysin genes nkla, nklb, nklc, and nkld.

6. The genetically-modified fish of claim 5, which has a genotype NK-lysin^{null/null}.

7. A genetically-modified fish whose genome is homozygous for a first engineered or induced genetic alteration in protein kinase, catalytic subunit-deficiency (prkdc), for a second engineered or induced genetic alteration in Nk-lysin, for a third engineered or induced genetic alteration in interleukin 2-receptor gamma a (il2rga), and for a fourth engineered or induced genetic alteration in interleukin 2-receptor gamma b (il2rgb);
wherein the first genetic alteration results in an inactivation of both alleles of prkdc;
wherein the second genetic alteration results in an inactivation of both alleles of Nk-lysin;
wherein the third genetic alteration results in an inactivation of both alleles of il2rga; and
wherein the fourth genetic alteration results in an inactivation of both alleles of il2rgb.

8. The genetically-modified fish of claim 7, which has a genotype of prkdc^{D3612fs}−/−; NK-lysin^{null/null}; il2rga^{Y91fs}−/−; il2rgb^{D33fs}−/−.

9. The genetically-modified fish of any one of claims 1 to 6, which is tolerant to irradiation.

10. A method of growing a mammalian cell, the method comprising transplanting the mammalian cell into the genetically-modified fish of any one of claims 1 to 9.

11. The method of claim 10, wherein the cell is a tumor cell.

12. The method of claim 10, wherein the cell is a stem cell or progeny of differentiated stem cell.

13. The method of claim 10, wherein the cell is a T cell.

14. The method of claim 10, wherein the cell is a blood cell.

15. A method of identifying a candidate therapeutic compound for the treatment of a mammalian tumor, the method comprising:
transplanting cells from a mammalian tumor into the genetically-modified fish of any one of claims 1 to 9;
contacting the fish with a test compound;
evaluating the growth of a tumor comprising the mammalian tumor cells in the presence of the test compound;
comparing the level of growth of the tumor in the presence of the test compound to a level of growth of a tumor in the presence of control substance; and
identifying a compound that decreases the level growth of the tumor as a candidate therapeutic compound.

16. The method of claim 15, wherein the mammalian tumor cells are from a subject with cancer, and the method further comprises administering the identified candidate therapeutic compound to the subject.

17. The method of claim 15 or 16, further comprising transplanting mammalian T cells into the genetically-modified fish.

18. The method of claim 16, wherein the therapeutic compound is a chimeric antigen receptor (CAR), a bispecific T cell engager (BiTE), or an antibody peptide epitope circuit therapy (APEC).

19. The method of claim 15, further comprising transplanting mammalian chimeric antigen receptor T cells into the genetically-modified fish.

20. A method of detecting an effect of a test compound on development of a cell or tissue, the method comprising:
transplanting a stem or progenitor cell into the genetically-modified fish of any of claims 1 to 9;
contacting the fish with a test compound for a time sufficient for the stem or progenitor cell to develop;
evaluating the development of the stem or progenitor cell or its progeny in the presence of the test compound;
comparing the development of the stem or progenitor cell or its progeny in the presence of the test compound to development of the stem or progenitor cell or its progeny in the absence of the test compound; and
identifying an effect of the test compound on development of the stem or progenitor cell or its progeny.

21. The method of claim 20, wherein the stem or progenitor cell is labeled.

22. The method of claim 20, wherein identifying an effect of the test compound on development of the stem or progenitor cell or its progeny comprises visualization of the cells in vivo or sectioning and staining the cells.

23. The method of claim 20, wherein the test compound is a drug or genetic modification.

24. The method of claim 20, wherein the stem or progenitor cell is a hematopoietic stem cell.


SEQUENCE LISTING (36 sequences)

SEQ ID NO: 1 - DNA, 33 bp, Danio rerio
tgcatatacagaaccgtatcaagcgcggggggt

SEQ ID NO: 2 - DNA, 27 bp, Danio rerio
taggcccttgactacatatggtgaggt

SEQ ID NO: 3 - DNA, 33 bp, Danio rerio
accccccgcgcttgatacggttctgtatatgca

SEQ ID NO: 4 - DNA, 27 bp, Danio rerio
acctcaccatatgtagtcaagggccta

SEQ ID NO: 5 - DNA, 27 bp, Danio rerio
tggttctggcattatctgtagtggaaa

SEQ ID NO: 6 - DNA, 28 bp, Danio rerio
ggtccgtcgtcgttcatcagctcgtcga

SEQ ID NO: 7 - DNA, 27 bp, Danio rerio
tttccactacagataatgccagaacca

SEQ ID NO: 8 - DNA, 28 bp, Danio rerio
tcgacgagctgatgaacgacgacggacc

SEQ ID NO: 9 - DNA, 53 bp, Danio rerio
tgtgcagtgtaaaatcatcaacgtggactatgtggagtgcatctggcaacgga

SEQ ID NO: 10 - DNA, 46 bp, Artificial Sequence
  Description of Artificial Sequence Synthetic oligonucleotide
tgtgcagtgtaaaatcatcaacgtgtggagtgcatctggcaacgga

SEQ ID NO: 11 - DNA, 20 bp, Artificial Sequence
  Description of Artificial Sequence Synthetic oligonucleotide
agaaccgtatcaagcgcggg

SEQ ID NO: 12 - DNA, 21 bp, Artificial Sequence
  Description of Artificial Sequence Synthetic oligonucleotide
ggcccttgactacatatggtg

SEQ ID NO: 13 - DNA, 20 bp, Artificial Sequence
  Description of Artificial Sequence Synthetic oligonucleotide
ggttctggcattatctgtag

SEQ ID NO: 14 - DNA, 20 bp, Artificial Sequence
  Description of Artificial Sequence Synthetic oligonucleotide
agcagcaagtagtcgagcag

SEQ ID NO: 15 - DNA, 18 bp, Danio rerio
tacattgaaaacaagcct

SEQ ID NO: 16 - DNA, 20 bp, Danio rerio
agtttggtgacggaacagga

SEQ ID NO: 17 - DNA, 18 bp, Danio rerio
tgygcagtgtaaaatcat

SEQ ID NO: 18 - DNA, 18 bp, Danio rerio
agtgcatctggcaacgga

SEQ ID NO: 19 - DNA, 13 bp, Danio rerio
gactgaattgctt

SEQ ID NO: 20 - DNA, 19 bp, Danio rerio
aagatttgggtcttacaga

SEQ ID NO: 21 - protein, 8 aa, Artificial Sequence
  Description of Artificial Sequence Synthetic peptide
AVSRLRAY

SEQ ID NO: 22 - protein, 8 aa, Artificial Sequence
  Description of Artificial Sequence Synthetic peptide
APPPVVLL

SEQ ID NO: 23 - protein, 5 aa, Artificial Sequence
  Description of Artificial Sequence Synthetic peptide
SGRSA

SEQ ID NO: 24 - protein, 9 aa, Artificial Sequence
  Description of Artificial Sequence Synthetic peptide
PRCFFRLGK

SEQ ID NO: 25 - protein, 8 aa, Artificial Sequence
  Description of Artificial Sequence Synthetic peptide
HPVGLLAR

SEQ ID NO: 26 - protein, 6 aa, Artificial Sequence
  Description of Artificial Sequence Synthetic peptide
PRGLRK

SEQ ID NO: 27 - protein, 6 aa, Artificial Sequence
  Description of Artificial Sequence Synthetic peptide
GPLPLR

SEQ ID NO: 28 - protein, 7 aa, Artificial Sequence
  Description of Artificial Sequence Synthetic peptide
AIPVSLR

SEQ ID NO: 29 - protein, 8 aa, Artificial Sequence
  Description of Artificial Sequence Synthetic peptide
GPLGVRGK

SEQ ID NO: 30 - protein, 5 aa, Artificial Sequence
  Description of Artificial Sequence Synthetic peptide
PRHLR

SEQ ID NO: 31 - protein, 9 aa, Artificial Sequence
  Description of Artificial Sequence Synthetic peptide
GPQGIAGQR

SEQ ID NO: 32 - protein, 7 aa, Artificial Sequence
  Description of Artificial Sequence Synthetic peptide
GKAFRRL

SEQ ID NO: 33 - protein, 5 aa, Artificial Sequence
  Description of Artificial Sequence Synthetic peptide
PRALM

SEQ ID NO: 34 - protein, 8 aa, Artificial Sequence
  Description of Artificial Sequence Synthetic peptide
DIKDIVGP

SEQ ID NO: 35 - protein, 8 aa, Artificial Sequence
  Description of Artificial Sequence Synthetic peptide
NPYRYTEI

SEQ ID NO: 36 - protein, 8 aa, Artificial Sequence
  Description of Artificial Sequence Synthetic peptide
RSSRDLLG